SEQUENCE LISTING (42 sequences)

SEQ ID NO: 1 - protein, 20 aa, Homo sapiens
KFASFIDKVRFLEQQNKMLE

SEQ ID NO: 2 - protein, 18 aa, Homo sapiens
LREYQELMNVKLALDIEI

SEQ ID NO: 3 - protein, 19 aa, Homo sapiens
KSYKVSTSGPRAFSSRSYT

SEQ ID NO: 4 - protein, 19 aa, Homo sapiens
KLALDIEIATYRKLLEGEE

SEQ ID NO: 5 - protein, 19 aa, Homo sapiens
RSNMDNMFESYINNLRRQL

SEQ ID NO: 6 - protein, 20 aa, Homo sapiens
LTDEINFLRQLYEEEIRELQ

SEQ ID NO: 7 - protein, 20 aa, Homo sapiens
KFASFIDKVRFLEQQNKVLE

SEQ ID NO: 8 - protein, 20 aa, Homo sapiens
KFASFIDKVRFLEQQNKLLE

SEQ ID NO: 9 - protein, 19 aa, Homo sapiens
KMALDIEIATYRKLLEGEE

SEQ ID NO: 10 - protein, 20 aa, Artificial Sequence
  Synthetic
  X (10)..(10): Where X is citrulline
KFASFIDKVXFLEQQNKMLE

SEQ ID NO: 11 - protein, 18 aa, Artificial Sequence
  Synthetic
  X (2)..(2): Where X is citrulline
LXEYQELMNVKLALDIEI

SEQ ID NO: 12 - protein, 19 aa, Artificial Sequence
  Synthetic
  X (11)..(11): Where X is citrulline
  X (16)..(16): Where X is citrulline
KSYKVSTSGPXAFSSXSYT

SEQ ID NO: 13 - protein, 19 aa, Artificial Sequence
  Synthetic
  X (12)..(12): Where X is citrulline
KLALDIEIATYXKLLEGEE

SEQ ID NO: 14 - protein, 19 aa, Artificial Sequence
  Synthetic
  X (1)..(1): Where X is citrulline
  X (16)..(16): Where X is citrulline
  X (17)..(17): Where X is citrulline
XSNMDNMFESYINNLXXQL

SEQ ID NO: 15 - protein, 20 aa, Artificial Sequence
  Synthetic
  X (9)..(9): Where X is citrulline
  X (17)..(17): Where X is citrulline
LTDEINFLXQLYEEEIXELQ

SEQ ID NO: 16 - protein, 19 aa, Mus musculus
KSYKMSTSGPRAFSSRSFT

SEQ ID NO: 17 - protein, 9 aa, Homo sapiens
YKVSTSGPR

SEQ ID NO: 18 - protein, 9 aa, Homo sapiens
IDKVRFLEQ

SEQ ID NO: 19 - protein, 9 aa, Homo sapiens
MFESYINNL

SEQ ID NO: 20 - protein, 9 aa, Homo sapiens
FLRQLYEEE

SEQ ID NO: 21 - protein, 9 aa, Homo sapiens
LMNVKLALD

SEQ ID NO: 22 - protein, 9 aa, Homo sapiens
IEIATYRKL

SEQ ID NO: 23 - protein, 19 aa, Artificial Sequence
  Synthetic
  X (11)..(11): Where X is citrulline
KSYKMSTSGPXAFSSRSFT

SEQ ID NO: 24 - protein, 483 aa, Homo sapiens
MSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 25 - protein, 513 aa, Homo sapiens
MNGVSWSQDLQEGISAWFGPPASTPASTMSIRVIQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 26 - protein, 490 aa, Mus musculus
MSIRVTQKSYKMSTSGPRAFSSRSFTSGPGARISSSSFSRVGSSSSSFRGSMGTGVGLGGFGGAGVGGITAVTVNQSLLSPLKLEVDPNIQAVRTQEKEQIKSLNNKFASFIDKVRFLEQQNKMLETKWSLLQQQKTSRSNMDNMFESYINNLRRQLEALGQEKLKLEAELGNMQGLVEDFKNKYEDEINKRTEMENEFVLIKKDVDEAYMNKVELESRLEGLTDEINFLRQIHEEEIRELQSQISDTSVVLSMDNSRSLDMDGIIAEVRAQYEDIANRSRAEAETMYQIKYEELQTLAGKHGDDLRRTKTEISEMNRNINRLQAEIEALKGQRASLEAAIADAEQRGEMAIKDAQTKLAELEAALQRAKQDMARQLREYQELMNVKLALDIEITTYRKLLEGEESRLESGMQNMSIHTKTTSGYSGGLSSSYGGLTSPGFSYGMSSFQPGFGSAGGSNTFSRTTKAVVVKKIETRDGKLVSESSDVVSK

SEQ ID NO: 27 - protein, 483 aa, Rattus rattus
MSVRVTQKSYKMSTSGPRAFSSRSFTSGPGARISSSSFSRVGSSSSSFRGSLGGFGGAGVGGITAVTVNQSLLNPLKLEVDPNIQAVRTQEKEQIKTLNNKFASFIDKVRFLEQQNKMLETKWSLLQQQKTSRSNMDNMFESYINNLRRQLEALGQEKLKLEVELGNMQGLVEDFKNKYEDEINKRTEMENEFVLIKKDVDEAYMNKVELESRLEGLTDEINFLRQIHEEEIRELQSQISDTSVVLSMDNSRSLDMDSIIAEVRAQYEEIANRSRAEAETMYQIKYEELQTLAGKHGDDLRRSKTEISEMNRNISRLQAEIDALKGQRATLEAAIADAEQRGELAVKDANAKLEDLKNALQKAKQDMARQLREYQELMNVKLALDIEIATYRKLLEGEESRLESGMQNMSIHTKTTSGYAGGLSSSYGGLTSPGFSYGMSSFQPGFGSVGGSSTYSRTKAVVVKKIETRDGKLVSESSDIMSK

SEQ ID NO: 28 - protein, 478 aa, Bos taurus
MSIRVTQKSYKVSTSAPRSFSSRSYTSGPGSRISSSAFSRVGSSSSFRGGLGTGMSMAGSYGGAPGLGGITAVTVNQSLLSPLKLEVDPNIQAVRTQEKEQIKTLNNKFASFIDKVRHLEQQNKVLETKWNLLQQQKTARSNIDNMFESYINNLRRQLETLAQEKLKLEVELGNMQGLVEDFKTKYEDEIQKRTDMENEFVIIKKDVDEAYMNKVELESRLEGLTDEINFYRQLYEEEIREMQSQISDTSVVLEMDNNRNLDLDGIIAEVKAQYEEIANRSRAEAEAMYQIKYEELQTLAGKHGDDLRRTKTEISEMNRNINRLQAEIEGLKGQRASLEAAIADAEQRGEMAVKDAQAKLAELEAALRNAKQDMARQLREYQELMNVKLALDVEIATYRKLLEGEESRLESGMQNMSIHTKTTSGYAGGLTSSYGTPGFNYSLSPGSFSRTSSKPVVVKKIETRDGKLVSESSDVLSK

SEQ ID NO: 29 - protein, 489 aa, Sus scrofa
MSIRVTQKSYKVSTSGPRSFSSRSYTSGPGTRISSSAFSRVGSSSSSFRGGLGSGMSLVGGYGGPSGLGGITAVTVNQSLLSPLKLEVDPNIQAVRTQEKEQIKTLNNKFASFIDKVRHLEQQNKVLETKWNLLQQQKTARSNIDNMFESYINNLRRQLETLAQEKLKLEVELGNMQGLVEDFKNKYEEEIQKRTDMENEFVIIKKDVDEAYMNKVELESRLEGLTDEINFYRQLYEEEIREMQSQISDTSVVLSMDNSRSLDLDGIIAEVKAQYEEIANRSRAEAETMYQIKYEELQSLAGKHGDDLRRTKTEISEMNRNISRLQAEIEGLKGQRASLEAAIADAEQRGELAVKDAQAKLAELEAALRTAKQDMARQLREYQELMNVKLALDVEIATYRKLLEGEESRLESGMQNMSIHTKTTSGYSGGVTYGTPGFNYSLSSYQSGGLGSVGGSGSFSRTSSKAVVVKKIETRDGKLVSESSDVLSK

SEQ ID NO: 30 - protein, 492 aa, Equus caballus
MSIRMTKSYRVSSVPRAFSSRSYTSAPGARISSSSFSRVGSSSSSFRSGLGSGMSLVGGYGGLGSAGGITAVTVNQSLLTPIKLEVDPNIQVVRTQEKEQIKTLNNKFASFIDKVRHLEQQNKILETKWSFLQQQKTARSNMDNMFESYINNLRRQLDALGQEKLKLEVELGNMQGLVEDFKNKYEDEINKRTEKENEFVLIKKDVDEAYMNKVELESRLEGLTDEINFFRQLYEEEIRELQSQISDTSVVLSMDNNRSLDLDGIIAEVKAQYEDIANRSRLEAEFMYQIKYEELQTLAGKHGDDLRRTKTEISETNRNINRLRAEIDGLKGQNAALEAAIADAEQRGELAIKDAQDKVTELEVALRTAKQDMARQLREYQELMNVKLALDVEIATYRKLLEGEESRLESGMQNLSIHTKTSSYSGGLSSTTFGGLTSPGLSYGLSSFQSSFSSGGSSGPIKLIGPPSAIVVKKIETRDGKLVSESSDILPK

SEQ ID NO: 31 - protein, 476 aa, Gallus gallus
MSVTITRKSVRSSSAVPVRSFSSRSFTAGPTVRMSAASAYGTLSGSRFGVGGLQRGPVAVSGGGIAAVTVNQSLLTPLNLDIDTSIQQVRKEEKEQIKTLNNRFASFIDKVRFLEQQNKMLETKWSLLQSQQPQRSDLRGLFEGYVGTLRRQLEGLGQERLRLEAELGSMQGLVEDFKNKYEEEINHRTEKENEFVLLKKDVDEAYMSKVELESRLESLTDEINFLRQLYDEELREMQSQISDTSVVLSMDNNRSLDLDSIIAEVKAQYEDIANRSRLEAETWYQSKYEELKTTAGKHGDDLRTTRSEISELNRMIQRIQAEIEVLKNQRATLETAIAEAEERGEMALKDARAKLSELEAALQKAKQDLARQLREYQELMNVKLALDIEIATYRKLLEGEESRLESGMQNLSIHTRTTGYSGGFGGGFGSSFSTGYTVTPPALESAAVPKSRAIVIKKIETRDGKLVSECADVLTN

SEQ ID NO: 32 - protein, 489 aa, Felis catus
MSIRVTQKSYKVSSSGPRGFSSLSFTSAPGSRISSALSRVGSGSSFRGGLGSSMSVVGGYGRPGGMGAITAVSVNQSLLSPLKLEVDPNIQAVRTQEKEQIKTLNNRFASFIDKVRHLEQQNKILETKWNLLQQQKTARSNMDNMFESYINNLRRQLDTLGQEKLKLEVELGNMQGLVEDFKNKYEEEIKERADMENEFVLIKKDVDEAYMNKVELESRLEGLTDEINFLRQLYEEEIRELQSQISDTSVVLSMDNSRSLDLDGIIAEVKAQYEEIANRSRAEAETMYQIKYEELQTLAGKHGDDLRRTKTEISEMNRNINRLQAEIEGLKNQRASLEAAVADAEQRGELAVKDANAKVAELEAALQRAKQDMARQLREYQELMNVKLALDIEIATYRKLLEGEESRLESGMQNMSIHTKTSTGYSGGLVSAYGGLMSPGLSYAMQSGFGSGGSSASSSRVSSSKAVVVKKIETRDGKLVSESSDVLSK

SEQ ID NO: 33 - protein, 491 aa, Canis familiaris
MSIRVTQKSYKMSTSSPRAFSSRSYTSGPSSRISSSAFSRVGSSSGSFRGGLNSSMSVVGGYGGPGVVGSITAVSVNQSLLNPLKLEVDPNIQAVRTQEKEQIKSLNNKFASFIDKVRHLEQQNKILETKWSLLQQQKTSRSNIDNMFESYINNLRRQLDTLGQEKLKLEVELGNMQGLVEDFKNKYEDEIKLRGDMENEFVLIKKDVDEAYMNKIELESRLEGLTDEINFLRQLYEEEIHELQSQISDTSVVLSMDNSRSLDLDGIIAEVKAQYEDIANRSRVEAETMYQIKYEELQTLAGKHGDDLRRTKTEISEMNRSISRLQAEIETLKNQRAALEAAIADAEQRGELAVKDANAKVAELEAALQRAKQDMARQLREYQELMNVKLALDIEIATYRKLLEGEESRLESGMQNMSIHTKTTSGYSGGLNLAYGGLTSPGLNYGQSSFQSGFGPGGSFSRSSSSKAVVVKKIETRDGKLVSESSDVLPK

SEQ ID NO: 34 - protein, 485 aa, Oryctolagus cuniculus
MSIRVTQKSYKVSTSGPRAFSSRSYTSAPGARISSSSFSRVGSSFRGAGMGLAGGYGGAPVGAGGITAVTVNQSLLSPLKLEVDPNIQAVRTQEKEQIKSLNNKFASFIDKVRFLEQQNKMLETKWSLLQQQKTARSNMDNMFESYINNLRRQLEALGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDGIIAEVRAQYEDIANRSRAEAESMYQVKYEELQLQAGKHGDDLRRTKTEISEMNRNISRIQAEIEALKGQRATLEAAIADAEQRGELAIKDANAKLAELEAALQRAKQDMARQLREYQELMNVKLALDIEIATYRKLLEGEESRLESGMQNMSIHTKTTTGYAGGLSSGFGSLASPGLNYGLSFQSGFGPGGGSSSFTRSKAVVVKKIETRDGKLVSESSDVLAK

SEQ ID NO: 35 - protein, 484 aa, Ovis aries
MSIRVTQKSYKVSTSAPRSFSSRSYTSGPGSRISSSAFSRVGSGSSFRGGLGTSMSMAGSYGGAPGLGGITAVTVNQSLLSPLKLEVDPNIQAVRTQEKEQIKTLNNKFASFIDKVRHLEQQNKVLETKWNLLQQQKTARSNIDNMFESYINSLRRQLETLAQDKLKLEVELGNMQGLVEDFKTKYEDEIQKRTDMENEFVIIKKDVDEAYMNKVELESRLEGLTDEINFYRQLYEEEIREMQSQISDTSVVLSMDNNRNLDLDGIIAEVKAQYEEIANRSRAEAETMYQIKYEELQTLAGKHGDDLRRTKTEISEMNRNISRLQAEIEGLKGQRASLEAAIADAEQRGEMAVKDAQAKLAELEAALRNAKQDMARQLREYQELMNVKLALDVEIATYRKLLEGEESRLESGMQNMSIHTKTTSGYAGGLTSAYGTPGFNYSLSLASGGGPGSFSRTSSKAVVVKKIETRDGKLVSESSDVLSK

SEQ ID NO: 36 - protein, 485 aa, Artificial Sequence
  Synthetic
MSIRVTQKSYKVSTSGPRAFSSRSYTSGPGSRISSSSFSRVGSSSSSFRGGLGSMSLGGYGGGLGGITAVTVNQSLLSPLKLEVDPNIQAVRTQEKEQIKTLNNKFASFIDKVRHLEQQNKMLETKWSLLQQQKTARSNMDNMFESYINNLRRQLETLGQEKLKLEVELGNMQGLVEDFKNKYEDEINKRTEMENEFVLIKKDVDEAYMNKVELESRLEGLTDEINFLRQLYEEEIRELQSQISDTSVVLSMDNSRSLDLDGIIAEVKAQYEDIANRSRAEAETMYQIKYEELQTLAGKHGDDLRRTKTEISEMNRNISRLQAEIEGLKGQRASLEAAIADAEQRGELAVKDANAKLAELEAALQRAKQDMARQLREYQELMNVKLALDIEIATYRKLLEGEESRLESGMQNMSIHTKTTSGYAGGLSSAYGGLSPGFSYLSQGFGSGGGSGSFSRTSSSKAVVVKKIETRDGKLVSESSDVLSK

SEQ ID NO: 37 - protein, 20 aa, Mus musculus
LTDEINFLRQIHEEEIRELQ

SEQ ID NO: 38 - protein, 19 aa, Mus musculus
KLALDIEITTYRKLLEGEE

SEQ ID NO: 39 - protein, 9 aa, Homo sapiens
PRAFSSRSY

SEQ ID NO: 40 - protein, 9 aa, Homo sapiens
LRQLYEEEI

SEQ ID NO: 41 - protein, 9 aa, Homo sapiens
REYQELMNV

SEQ ID NO: 42 - protein, 19 aa, Mus musculus
  X (11)..(11): Where X is citrulline
  X (16)..(16): Where X is citrulline
KSYKVSTSGPXAFSSXSYT

(19) United States
(12) Patent Application Publication
(10) Pub. No.: US 2025/0269000 A1
(43) Pub. Date: Aug. 28, 2025

(54) ANTI-TUMOUR RESPONSES TO CYTOKERATINS

(71) Applicant: SCANCELL LIMITED, Oxford, Oxfordshire (GB)

(72) Inventors: Linda Gillian Durrant, Oxford, Oxfordshire (GB); Victoria Anne Brentville, Oxford, Oxfordshire (GB); Katherine Cook, Oxford, Oxfordshire (GB); Peter Symonds, Oxford, Oxfordshire (GB)

(21) Appl. No.: 18/253,653

(22) PCT Filed: Nov. 22, 2021
(86) PCT No.: PCT/EP2021/082541

§ 371 Date: May 19, 2023

(30) Foreign Application Priority Data
Nov. 23, 2020 (GB) 2018395.0

(51) Int. Cl. A61K 39/00; A61K 39/385; A61P 35/00; C07K 14/47; C07K 16/18; G01N 33/50; G01N 33/569; G01N 33/68

(52) U.S. Cl. CPC A61K 39/0011; A61K 39/385; A61P 35/00; C07K 14/4741; C07K 16/18; G01N 33/505; G01N 33/56972; G01N 33/6881; A61K 2039/605; G01N 2333/4742

(57) ABSTRACT

The present invention relates to citrullinated Cytokeratin peptides that can be used in cancer immunotherapy. The modified peptides may be used as vaccines or as targets for T cell receptor (TCR) and adoptive T cell transfer therapies. Such vaccines or targets may be used in the treatment of cancer.

The present invention relates to modified Cytokeratin peptides that can be used in cancer immunotherapy. The modified peptides may be used as vaccines or as targets for T cell receptor (TCR) and adoptive T cell transfer therapies. Such vaccines or targets may be used in the treatment of cancer.

In order to be effective, cancer vaccines need to induce a potent immune response that is able to break the tolerance and overcome the immunosuppressive tumour environment. The importance of CD4 T cells in mediating tumour destruction has been recently highlighted, however, the induction of self-specific CD4 responses has proved more difficult. In contrast, CD4 T cells recognising modified self-epitopes have been shown to play a role in the pathophysiology of several autoimmune diseases such as rheumatoid arthritis (RA), collagen II-induced arthritis, sarcoidosis, celiac disease and psoriasis (Choy 2012; Grunewald and Eklund 2007; Coimbra et al. 2012; Holmdahl et al. 1985). One of these common modifications is the citrullination of arginine, which involves the conversion of the positively charge aldimine group (═NH) of arginine to the neutrally charged ketone group (═O) of citrulline. Citrullination is mediated by Peptidylarginine deiminases (PADs), which are a family of calcium dependent enzymes found in a variety of tissues. A recent report by Ireland et al. (Ireland and Unanue 2011), demonstrated that the presentation of citrullinated T cell epitopes on antigen presenting cells (APCs) is also dependent upon autophagy and PAD activity. This process has also been demonstrated to be an efficient mechanism to enable processing of endogenous antigens for presentation on MHC class II molecules on professional APCs as well as epithelial cells (Munz 2012; Schmid, Pypaert, and Munz 2007). Autophagy is constitutive in APCs, but in other cells it is only induced by stress (Green and Levine 2014). T cells recognising citrullinated epitopes do not target normal healthy cells that do not express citrullinated peptides. Autophagy is triggered by stress such as hypoxia and nutrient starvation and is upregulated to promote tumour survival (Green and Levine 2014).

Cytokeratins are the largest family of intermediate filament (IF) proteins that are expressed on all epithelial cells, they have significant biochemical diversity (Liao, Ku, and Omary 1997), wide tissue distribution, multiple functions and disease associations (Chou, Skalli, and Goldman 1997; Chang et al. 2013). They play important roles in maintaining shapes and rigidity of the cells by forming cytoplasmic scaffold that emanates from the plasma membrane (Fuchs and Cleveland 1998). In addition to structural functions, they are also involved in cell signalling pathways that regulate cell cycle progression, apoptosis, cellular response to stress, protein synthesis, cell size and membrane trafficking (Paramio and Jorcano 2002; Coulombe and Omary 2002; Oshima 2002).

The profiling of cytokeratin expression allows classification of epithelial cells according to the presence of specific cytokeratins (Moll, Divo, and Langbein 2008; Moll et al. 1982). Cytokeratin 8, 18 and 19 are expressed on simple epithelial cells, whereas cytokeratin 5 and 14 are expressed on basal epithelial cells. Cytokeratin filaments are flexible and can reorganise in response to changes in mechanical and non-mechanical stimuli to regulate different cellular processes, including cell signalling and migration (Gu and Coulombe 2007; Chung, Rotty, and Coulombe 2013).

The expression of cytokeratins has been used by pathologists to help classify different cancer types; the majority of cancer cells originate from epithelial cells. Staining tumours for the expression of cytokeratin proteins has proven invaluable to pathologists in helping to identify tumours; since the 1980s cytokeratin specific monoclonal antibodies have been used to diagnose cancers (Oshima 2007; Moll, Divo, and Langbein 2008). For example, for non-small cell lung cancer (NSCLC), the overexpression of cytokeratin 17 is associated with squamous cell carcinoma when compared to adenocarcinomas (Moll, Divo, and Langbein 2008).

An increasing number of studies have shown that cytokeratins play a role in cancer cell metastasis and contribute to patient prognosis (Karantza 2011). For example, in colorectal cancer, a reduced expression of cytokeratin 8 and cytokeratin 20 has been associated with epithelial-to-mesenchymal (EMT) cancer cell transition, and a decrease in patient survival (Knosel et al. 2006). In pancreatic cancer patients the expression of cytokeratin 20 in the bone marrow and/or blood in patients with pancreatic adenocarcinomas correlate with a poor prognosis (Soeth et al. 2005; Matros et al. 2006; Schmitz-Winnenthal et al. 2006). The co-expression of cytokeratin 7 and cytokeratin 19 in clear-cell RCC is associated with a better clinical outcome (Mertz et al. 2008). However, the co-expression of cytokeratin 8 and 18 on circulating tumour cells correlates with the presence of metastases at the time of primary tumour resection and poor overall survival (Bluemke et al. 2009). Other correlations with cytokeratin expression and prognosis have also been seen in many other cancers including gastric cancer (Katsuragi et al. 2007), hepatocellular carcinoma (Yang et al. 2008), endometrial cancer (Stefansson, Salvesen, and Akslen 2006) and skin cancer (Chen et al. 2009).

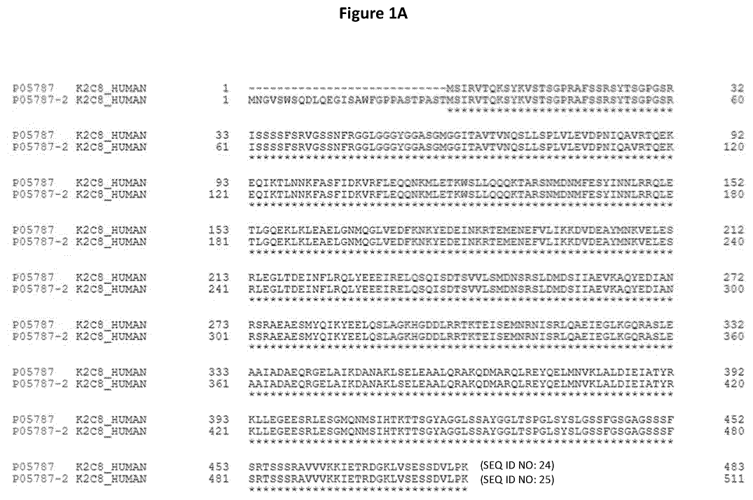

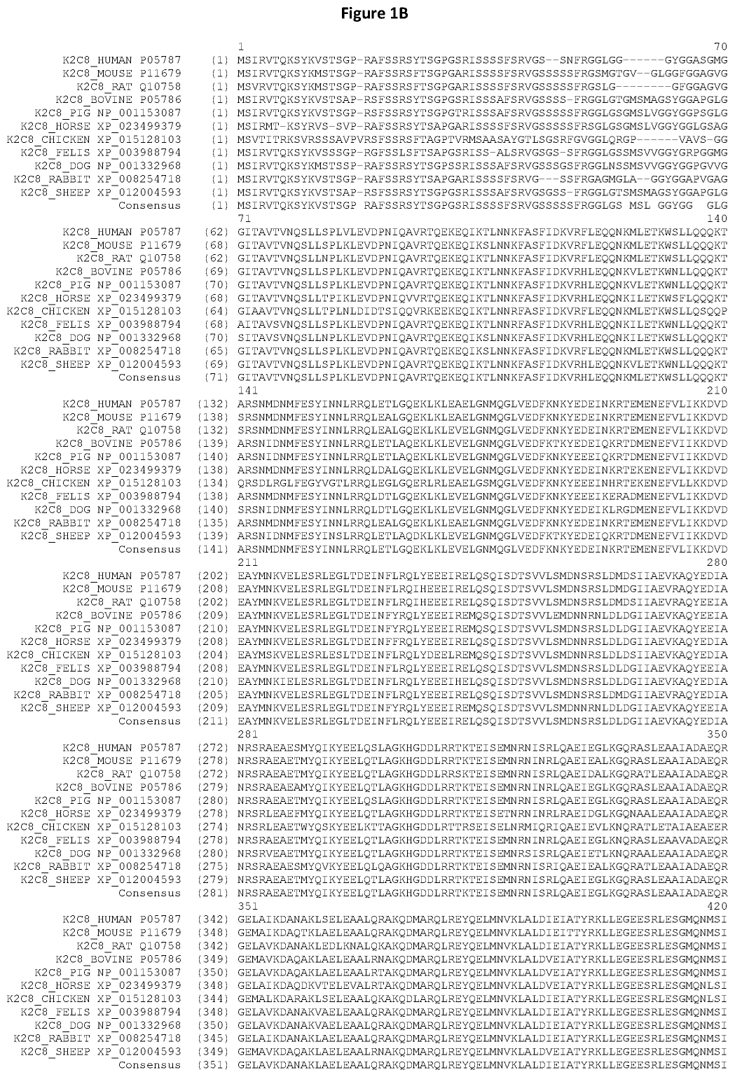

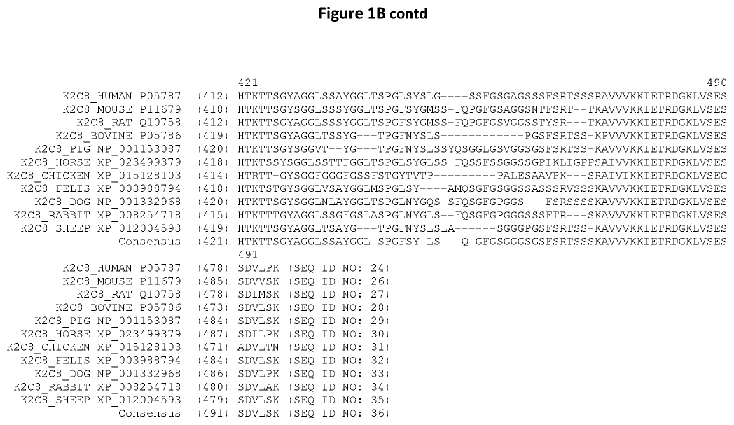

There are 54 keratin genes with 28 type I and 26 type II sequences that are regulated so that heterodimers of Type I and II cytokeratins form that further polymerise to form filaments. This process is regulated at the transcriptional and post-translational level (Gu and Coulombe 2007). Cytokeratin 8, also known as Keratin, type II cytoskeletal 8, keratin 8 (KRT8, CK8, K8), is a member of the type II cytokeratin family local on chromosome 12. Two alternatively spliced variants are produced by alternative promoter usage and alternative splicing (FIG. 1A), isoform 1 (P05787-1) which is 53 kDa, 483 amino acids and isoform 2 (P05787-2) which is 56 kDa, 511 amino acids. A number of mutations have been also identified and associated with disease of the liver, pancreatitis and inflammatory bowel disease (Szeverenyi et al. 2008).

Cytokeratin 8 (Cyk8) is a well-known epithelial marker protein that polymerises with Cytokeratin 18. This cytokeratin pair is the first to be expressed in embryogenesis. In adult tissues, the expression of this pair is restricted to simple (such as liver, pancreas, kidney) and mixed (such as breast, lung) epithelia (Moll et al. 1982; Owens and Lane 2003; Franke et al. 1981; Blobel et al. 1984). Over-expression of this pair has been observed in adenocarcinomas and squamous cell carcinomas (Oshima, Baribault, and Caulin 1996; Vaidya et al. 1989). It has been reported in breast cancer and melanoma that Cytokeratin 8 and 18 expression along with vimentin results in an increase in drug resistance, invasion and metastasis in breast cell carcinomas and melanomas (Thomas et al. 1999). Aberrant expression of Cyk8 is found in non-small-cell lung cancer and also present in the sera of patients with NSCLC (Fukunaga et al. 2002). Autoantibodies of Cyk8 have also been found in patients with rheumatoid arthritis (RA) and described as one of the real antigens of the so called anti-keratin antibodies associated with RA (Wang et al. 2015).

The cytokeratin proteins play an important role in maintaining epithelial structural integrity particularly during stress. They are key cellular regulators but increasing evidence shows they play a role in epithelial tumorigenesis and cancer treatment responsiveness. Post-translational modifications of proteins occurs under conditions of cellular stress. One such modification involves citrullination, the conversion of arginine residues to citrulline by peptidylarginine deiminase (PAD) enzymes. Citrullination occurs as a result of a degradation and recycling process (autophagy) that is induced in stressed cells (Ireland and Unanue 2011). Citrullinated epitopes can subsequently be presented on MHC class II molecules for recognition by CD4 T cells. The potent immune responses unleashed in response to citrullinated proteins can be harnessed and redirected to destroy cancer cells. This immune response is mediated by killer CD4 T cells that then secrete high amounts of IFNγ. This increases MHC class II expression and then directly kills the tumour cells, without the need for CD8 T cell involvement (Brentville et al. 2016; Durrant, Metheringham, and Brentville 2016). Tumour recognition depends upon both citrullination and autophagy. A high number of IFNγ secreting CD4 T cells have been shown to be induced following immunisation of mice with two citrullinated peptides derived from the cytoskeletal protein, vimentin. Ex-vivo, these CD4 T cells recognise tumour cells in which autophagy is induced by either starvation or rapamycin. Vimentin's function and expression in tumours has been detailed previously in WO2014023957.

According to a first aspect of the invention, there is provided a citrullinated T cell antigen comprising, consisting essentially of or consisting of,
(i) one or more of the amino acid sequences:

(SEQ ID NO: 1)
KFASFIDKVRFLEQQNKMLE
(Cyk8 101-120)
(SEQ ID NO: 2)
LREYQELMNVKLALDIEI
(Cyk8 371-388)
(SEQ ID NO: 16)
KSYKMSTSGPRAFSSRSFT
(Murine Cyk8 8-26)
(SEQ ID NO: 3)
KSYKVSTSGPRAFSSRSYT
(Cyk8 8-26)
(SEQ ID NO: 4)
KLALDIEIATYRKLLEGEE
(Cyk8 381-399)
(SEQ ID NO: 5)
RSNMDNMFESYINNLRRQL
(Cyk8 133-151)
and
(SEQ ID NO: 6)
LTDEINFLRQLYEEEIRELQ
(Cyk8 217-236)

wherein at least one arginine (R) residue in the sequence is replaced with citrulline, and/or
(ii) one or more amino acid sequences of i), with the exception of 1, 2 or 3 amino acid substitutions, and/or 1, 2 or 3 amino acid insertions, and/or 1, 2 or 3 amino acid deletions in a non-arginine position.

The inventors have unexpectedly found that it is possible to raise T cell responses to certain antigens from Cytokeratin 8 expressed on tumour cells in which at least one arginine has been replaced by citrulline. Furthermore, citrulline-containing peptides permit the development of T cell-based therapies, including but not limited to tumour vaccines, as well as T cell receptor (TCR) and adoptive T cell transfer therapies. The inventors have shown that in normal donors, cancer patients and HLA transgenic mice, there is a repertoire of T cells which recognise citrullinated cytokeratin peptides and produce IFNγ. They have also stimulated an immune response to a tumour that expresses citrullinated cytokeratin epitopes KFASFIDKVRFLEQQNKMLE (SEQ ID NO: 1) (Cyk8 101-120) and/or LREYQELMNVKLALDIEI (SEQ ID NO: 2) (Cyk8 371-388) in a subject by administering the citrullinated epitope(s).

The T cell antigen of the present invention may be a MHC class I or class II antigen, i.e. form a complex with and be presented on a MHC class I or II molecule respectively. The skilled person can determine whether or not a given polypeptide forms a complex with an MHC molecule by determining whether the MHC can be refolded in the presence of the polypeptide. If the polypeptide does not form a complex with MHC, the MHC will not refold properly. Refolding is commonly confirmed using an antibody that recognises MHC in a folded state only. Further details can be found in (Garboczi, Hung, and Wiley 1992).

It is preferred if all of the arginine amino acid residues in the antigen may be converted to citrulline. 1, 2 or 3 of the arginine amino acid residues in the antigen may be converted to citrulline, with the remainder being unconverted. Thus, an antigen of the present invention may have 1, 2 or 3 citrulline residues. Antigens of the present invention may be up to 25 amino acids in length. They may be at least 5 amino acids in length. They may be no longer than 18, 19, 20, 21, 22, 23 or 24 amino acids. The T cell antigen of the present invention may be tumour-associated and may stimulate an immune response against the tumour.

The inventors have found a high degree of sequence homology between the peptides identified from Cytokeratin 8 and other Cytokeratins or similar proteins that contain the same or similar peptides, as such the amino acid sequences.
KFASFIDKVRFLEQQNKMLE (SEQ ID NO: 1) (Cyk8 101-120) is also contained within Cytokeratin 2 with an amino acid substitution at position 18, the sequence essentially consisting of KFASFIDKVRFLEQQNKVLE (SEQ ID NO: 7)
KFASFIDKVRFLEQQNKMLE (SEQ ID NO: 1) (Cyk8 101-120) is also contained within Cytokeratin 7 with an amino acid substitution at position 18, the sequence essentially consisting of KFASFIDKVRFLEQQNKLLE (SEQ ID NO: 8)
KLALDIEIATYRKLLEGEE (SEQ ID NO: 4) (Cyk8 381-399) is also contained within Cytokeratin 4 and is identical.
KLALDIEIATYRKLLEGEE (SEQ ID NO: 4) (Cyk8 381-399) is also contained within Vimentin with an amino acid substitution at position 2, the sequence essentially consisting of KMALDIEIATYRKLLEGEE (SEQ ID NO: 9)
KLALDIEIATYRKLLEGEE (SEQ ID NO: 4) (Cyk8 381-399) is also contained within Glial fibrillary protein and is identical.

The inventors have shown that, in normal healthy donors and HLA transgenic mice, T cells recognising citrullinated Cyk8 peptides produce IFNγ and can be detected following stimulation with Cyk8 peptides. They have also shown that certain citrullinated Cyk8 peptides generate a T cell response in vivo and, as such, can be used as a vaccine target for cancer therapy.

The T cell antigen of the present invention may comprise, consist essentially of, or consist of i) one or more of the following amino acid sequences:
KFASFIDKVRFLEQQNKMLE (SEQ ID NO: 1)
LREYQELMNVKLALDIEI (SEQ ID NO: 2)
KSYKMSTSGPRAFSSRSFT (SEQ ID NO: 16)
KSYKVSTSGPRAFSSRSYT (SEQ ID NO: 3)
KLALDIEIATYRKLLEGEE (SEQ ID NO: 4)
RSNMDNMFESYINNLRRQL (SEQ ID NO: 5) and
LTDEINFLRQLYEEEIRELQ (SEQ ID NO: 6) and/or
ii) one or more of the amino acid sequences of i), with the exception of 1, 2 or 3 amino acid substitutions, and/or 1, 2 or 3 amino acid insertions, and/or 1, 2 or 3 amino acid deletions in a non-arginine position. The antigen may have a total of 1, 2, 3, 4 or 5 amino acid modifications selected from substitutions, insertions and substitutions in a non-arginine position. The T cell antigen of ii) is preferably capable of raising an immune response against tumours including, but not restricted to, thyroid, colorectal, urothelial, stomach, liver, carcinoid, pancreatic, renal, prostate, lung, breast and gynaecological tumours

It is preferred if the T cell antigen of the present invention comprises, consists essentially of, or consists of i) one or more of the following amino acid sequences:

(SEQ ID NO: 10)
KFASFIDKV-cit-FLEQQNKMLE
(Cyk8 101-120)
(SEQ ID NO: 11)
L-cit-EYQELMNVKLALDIEI
(Cyk8 371-388)
(SEQ ID NO: 42)
KSYKMSTSGP-cit-AFSS-cit-SFT
(Murine Cyk8 8-26)
(SEQ ID NO: 12)
KSYKVSTSGP-cit-AFSS-cit-SYT
(Cyk8 8-26)
(SEQ ID NO: 13)
KLALDIEIATY-cit-KLLEGEE
(Cyk8 381-399)
(SEQ ID NO: 14)
cit-SNMDNMFESYINNL-cit-cit-QL
(Cyk8 133-151)
(SEQ ID NO: 15)
LTDEINFL-cit-QLYEEEI-cit-ELQ
(Cyk8 217-236)

wherein “cit” represents citrulline, and/or
ii) the amino acid sequence of i), with the exception of 1, 2 or 3 amino acid substitutions, and/or 1, 2 or 3 amino acid insertions, and/or 1, 2 or 3 amino acid deletions in a non-citrulline position. The antigen may have a total of 1, 2, 3, 4 or 5 amino acid modifications selected from substitutions, insertions and substitutions in a non-arginine position. The T cell antigen of ii) is preferably capable of raising an immune response against tumours including, but not restricted to, thyroid, colorectal, urothelial, stomach, liver, carcinoid, pancreatic, renal, prostate, lung, breast and gynaecological tumours.

The inventors have unexpectedly found that certain citrullinated peptides derived from Cytokeratin 8 can be used to raise an immune response against tumours including, but not restricted to, thyroid, colorectal, urothelial, stomach, liver, carcinoid, pancreatic, renal, prostate, lung, breast and gynaecological tumours. The inventors have shown that
KSYKVSTSGP-cit-AFSS-cit-SYT (SEQ ID NO: 12) (Cyk8 8-26, citrullinated at positions 18 and 23)
KFASFIDKV-cit-FLEQQNKMLE (SEQ ID NO: 10) (Cyk8 101-120, citrullinated at position 110)
KLALDIEIATY-cit-KLLEGEE (SEQ ID NO: 13) (Cyk8 381-399, citrullinated at position 392)
cit-SNMDNMFESYINNL-cit-cit-QL (SEQ ID NO: 14) (Cyk8 133-151, citrullinated at position 133, 148 and 149)
L-cit-EYQELMNVKLALDIEI (SEQ ID NO: 11) (Cyk8 371-388, citrullinated at position 372)
LTDEINFL-cit-QLYEEEI-cit-ELQ (SEQ ID NO: 15) (Cyk8 217-236, citrullinated at positions 225 and 233)
   generated an immune response in vivo to citrullinated Cyk8 epitopes. The peptides KFASFIDKV-cit-FLEQQNKMLE (SEQ ID NO: 10) (Cyk8 101-12), L-cit-EYQELMNVKLALDIEI (SEQ ID NO: 11) (Cyk8 371-388) and cit-SNMDNMFESYINNL-cit-cit-QL (SEQ ID NO: 14) (Cyk8 133-151) are homologous to mouse. The peptides KSYKVSTSGP-cit-AFSS-cit-SYT (SEQ ID NO: 12) (Cyk8 8-26), KLALDIEIATY-cit-KLLEGEE (SEQ ID NO: 13) (Cyk8 381-399) and LTDEINFL-cit-QLYEEEI-cit-ELQ (SEQ ID NO: 15) (Cyk8 217-236) are not homologous to mouse with 2, 1 and 2 amino acid mismatches respectively.

Citrullinated peptides are known to stimulate T cell responses in autoimmune patients with the shared HLA-DR4 motif. In contrast, the inventors are the first to show that certain citrullinated Cyk8 peptides, such as Cyk8 101-120 (cit at position 110), Cyk8 133-151 (cit at position 133, 148 and 149), Cyk8 217-236 (cit at position 225), Cyk8 371-388 (cit at position 372), Cyk8 381-399 (cit at position 392) and Cyk8 366-385 (cit at position 372) can stimulate potent T cell responses in HLA-DP4 transgenic mice. In addition, Cyk8 8-26 cit can stimulate a potent CD4 T cell response in C57BL/6 mice. As HLA-DP4 is expressed by 70% of the population, this makes it a promising vaccine for the treatment of haematological and solid tumours. Some healthy donors showing responses to Cyk8 101-120 cit citrullinated at position 110 expressed HLA-DP4, however, some donors that showed a response were not HLA-DP4 positive, indicating that other HLA class II alleles could also be presenting this peptide. The response to Cyk8 101-120 cit, Cyk8 133-151 cit, Cyk8 217-236 cit, Cyk8 371-388 cit and Cyk8 381-399 cit showed minimal reactivity to the unmodified wildtype sequence. T cells recognising Cyk8 371-388, Cyk8 101-120, Cyk8 8-26 citrullinated peptide antigens can target tumour cells and elicit strong anti-tumour effects in vivo, thus providing the first evidence for the use of citrullinated Cyk8 371-388, Cyk8 101-120, Cyk8 8-26 as a vaccine target for cancer therapy.

The MHC class II antigen processing pathway can be influenced by many factors, such as the internalisation and processing of exogenous antigen, the peptide binding motif for each MHC class II molecule and the transportation and stability of MHC class II: peptide complex. The MHC class II peptide binding groove is open at both ends and it is less constrained by the length of the peptide compared to MHC Class I molecules. The peptides that bind to MHC class II molecules range in length from 13-25 amino acids long and typically protrude out of the MHC molecule (Kim et al. 2014; Sette et al. 1989). These peptides contain a consecutive stretch of nine amino acids, referred to as the core region. Some of these amino acids interact directly with the peptide binding groove (Andreatta et al. 2017). The amino acids either side of the core peptide protrude out of the peptide binding groove; these are known as peptide flanking regions. They can also impact peptide binding and subsequent interactions with T cells (Arnold et al. 2002; Carson et al. 1997; Godkin et al. 2001).

MHC class II molecules are highly polymorphic, the peptide binding motifs are highly degenerate with many promiscuous peptides having been identified that can bind multiple MHC class II molecules (Consogno et al. 2003). The amino acids that are critical for peptide binding have been identified from crystallography studies of MHC class II: peptide complexes (Corper et al. 2000; Dessen et al. 1997; Fremont et al. 1996; Ghosh et al. 1995; Latek et al. 2000; Li et al. 2000; Lee, Wucherpfennig, and Wiley 2001; Brown et al. 1993; Smith et al. 1998; Stern et al. 1994; Scott et al. 1998; Fremont et al. 1998). These studies have indicated that P1, P4, P6 and P9 always point towards the MHC whereas P-1, P2, P5, P8 and P11 always orient towards the TCR. The frequency of HLA-DR and HLA-DP alleles is listed in Table 1 (Thomsen and Nielsen 2012; Gonzalez-Galarza et al. 2015).

TABLE 1
HLA-DR and DP allele frequency in the UK population

| | % individuals that have | |
|---|---|---|
| Allele | the allele | Sample Size |
| DRB1*04 | 32 | 57,732 |
| DRB1*03 | 28 | 57,732 |
| DRB1*07 | 28 | 57,732 |
| DRB1*15 | 28 | 57,732 |
| DBR1*01 | 22 | 57,732 |
| DPB1*0401 | 70 | 187 |
| DPB1*0301 | 22 | 187 |
| DPB1*0201 | 20 | 187 |
| DPB1*0101 | 14 | 187 |

In contrast, MHC class I molecules show more restricted peptide binding properties. Amino acids critical for binding to MHC class I have also been identified through prediction algorithms analysing known naturally binding peptides (Jurtz et al. 2017), which indicated that (with the exception of HLA-B*0801) P2 and P9 orient towards the MHC acting as binding anchor residues.

Cytokeratin 8 is highly conserved between those species (FIG. 1B) in which the gene has been cloned (chicken, mouse, dog, sheep, cow, horse, pig and human). Accordingly, an antigen of the invention, optionally in combination with a nucleic acid comprising a sequence that encodes such an antigen, can be used for treating cancer in non-human mammals.

The invention also includes within its scope peptides having the amino acid sequence as set out above and sequences having substantial identity thereto, for example, at least 70%, at least 80%, at least 85%, at least 90%, at least 95% or at least 99% identity thereto, as well as their use in medicine and in particular in a method for treating cancer. Such peptides are preferably capable of raising an immune response against tumours including, but not restricted to, thyroid, colorectal, urothelial, stomach, liver, carcinoid, pancreatic, renal, prostate, lung, breast and gynaecological tumours. The percent identity of two amino acid sequences or of two nucleic acid sequences is generally determined by aligning the sequences for optimal comparison purposes (e.g., gaps can be introduced in the first sequence for best alignment with the second sequence) and comparing the amino acid residues or nucleotides at corresponding positions. The “best alignment” is an alignment of two sequences that results in the highest percent identity. The percent identity is determined by comparing the number of identical amino acid residues or nucleotides within the sequences (i.e. % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (Karlin and Altschul 1993). The NBLAST and XBLAST programs of Altschul, et al. have incorporated such an algorithm (Altschul et al. 1990). BLAST nucleotide searches can be performed with the NBLAST program (score=100, word length=12) to obtain nucleotide sequences homologous to a nucleic acid molecules of the invention. BLAST protein searches can be performed with the XBLAST program (score=50, word length=3) to obtain amino acid sequences homologous to a protein molecules of the invention. To obtain gapped alignments for comparison purposes, Gapped BLAST can be utilized as described in (Altschul et al. 1997). Alternatively, PSI-Blast can be used to perform an iterated search that detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller (Myers and Miller 1989). The ALIGN program (version 2.0) which is part of the GCG sequence alignment software package has incorporated such an algorithm. Other algorithms for sequence analysis known in the art include ADVANCE and ADAM as described in (Torelli and Robotti 1994) and FASTA described in (Pearson and Lipman 1988). Within FASTA, ktup is a control option that sets the sensitivity and speed of the search.

Amino acid substitution means that an amino acid residue is substituted for a replacement amino acid residue at the same position. Inserted amino acid residues may be inserted at any position and may be inserted such that some or all of the inserted amino acid residues are immediately adjacent one another or may be inserted such that none of the inserted amino acid residues is immediately adjacent another inserted amino acid residue.

The antigen of the invention may comprise one, two or three additional amino acids at the C terminal end and/or at the N-terminal end thereof. An antigen of the invention may comprise the amino acid sequence set out above with the exception of one amino acid substitution and one amino acid insertion, one amino acid substitution and one amino acid deletion, or one amino acid insertion and one amino acid deletion. An antigen of the invention may comprise the amino acid sequence set out above, with the exception of one amino acid substitution, one amino acid insertion and/or one amino acid deletion.

Inserted amino acids and replacement amino acids may be naturally occurring amino acids or may be non-naturally occurring amino acids and, for example, may contain a non-natural side chain. Such altered peptide ligands are discussed further in (Douat-Casassus et al. 2007; Hoppes et al. 2014) and references therein). If more than one amino acid residue is substituted and/or inserted, the replacement/inserted amino acid residues may be the same as each other or different from one another. Each replacement amino acid may have a different side chain to the amino acid being replaced.

Preferably, antigens of the invention bind to MHC in the peptide binding groove of the MHC molecule. Generally, the amino acid modifications described above will not impair the ability of the peptide to bind MHC. In a preferred embodiment, the amino acid modifications improve the ability of the peptide to bind MHC. For example, mutations may be made at positions which anchor the peptide to MHC. Such anchor positions and the preferred residues at these locations are known in the art.

An antigen of the invention may be used to elicit an immune response, e.g. a T cell response. If this is the case, it is important that the immune response is specific to the intended target in order to avoid the risk of unwanted side effects that may be associated with an “off target” immune response. Therefore, it is preferred that the amino acid sequence of a polypeptide of the invention does not match the amino acid sequence of a peptide from any other protein(s), in particular, that of another human protein. A person of skill in the art would understand how to search a database of known protein sequences to ascertain whether an antigen according to the invention is present in another protein.

The invention describes an in vitro method of screening to identify a citrullinated T cell epitope of a target peptide that stimulates anti-tumour immunity, comprising: screening the target peptide for induction of a T cell response specific to a citrullinated epitope; and screening T cells specific for the citrullinated epitope for tumour recognition. Screening for induction of T cell response to a citrullinated epitope may comprise sorting CD4 and CD8 T cells to identify whether the citrullinated epitope is a CD4 or CD8 epitope. The target peptide may be cytokeratin and more specifically cytokeratin8.

Antigens of the invention can be synthesised easily by Merrifield synthesis, also known as solid phase synthesis, or any other peptide synthesis methodology. GMP grade polypeptide is produced by solid-phase synthesis techniques by Multiple Peptide Systems, San Diego, CA. Alternatively, the peptide may be recombinantly produced, if so desired, in accordance with methods known in the art. Such methods typically involve the use of a vector comprising nucleic acid sequence encoding the polypeptide to be expressed, to express the polypeptide in vivo; for example, in bacteria, yeast, insect or mammalian cells. Alternatively, in vitro cell free systems may be used. Such systems are known in the art and are commercially available for example from Life Technologies, Paisley, UK. The antigens may be isolated and/or may be provided in substantially pure form. For example, they may be provided in a form which is substantially free of other polypeptides or proteins. Peptides of the invention may be synthesised using Fmoc chemistry or other standard techniques known to those skilled in the art.

In a second aspect, the invention provides a complex of the antigen of the first aspect and an MHC molecule. Preferably, the antigen is bound to the peptide binding groove of the MHC molecule. The MHC molecule may be MHC class I or II. The MHC class II molecule may be a DP, DR or DQ allele, such as HLA-DR4, DR1, DP4, DP2, DP5, DQ2, DQ3, DQ5 and DQ6. HLA-DP4 is preferred. The MHC class I molecule may be a A or B allele.

The antigen and complex of the invention may be isolated and/or in a substantially pure form. For example, the antigen and complex may be provided in a form which is substantially free of other polypeptides or proteins. It should be noted that in the context of the present invention, the term “MHC molecule” includes recombinant MHC molecules, non-naturally occurring MHC molecules and functionally equivalent fragments of MHC, including derivatives or variants thereof, provided that peptide binding is retained. For example, MHC molecules may be fused to a therapeutic moiety, attached to a solid support, in soluble form, and/or in multimeric form.

Methods to produce soluble recombinant MHC molecules with which antigens of the invention can form a complex are known in the art. Suitable methods include, but are not limited to, expression and purification from E. coli cells or insect cells. Alternatively, MHC molecules may be produced synthetically, or using cell free systems.

Antigens and/or antigen-MHC complexes of the invention may be associated with a moiety capable of eliciting a therapeutic effect. Such a moiety may be a carrier protein which is known to be immunogenic. KLH (keyhole limpet hemocyanin) is an example of a suitable carrier protein used in vaccine compositions. Alternatively, the antigens and/or antigen-MHC complexes of the invention may be associated with a fusion partner. Fusion partners may be used for detection purposes, or for attaching said antigen or MHC to a solid support, or for MHC oligomerisation. The MHC complexes may incorporate a biotinylation site to which biotin can be added, for example, using the BirA enzyme. Other suitable fusion partners include, but are not limited to, fluorescent, or luminescent labels, radiolabels, nucleic acid probes and contrast reagents, antibodies, or enzymes that produce a detectable product. Detection methods may include flow cytometry, microscopy, electrophoresis or scintillation counting.

Antigen-MHC complexes of the invention may be provided in soluble form or may be immobilised by attachment to a suitable solid support. Examples of solid supports include, but are not limited to, a bead, a membrane, sepharose, a magnetic bead, a plate, a tube, a column.

Antigen-MHC complexes may be attached to an ELISA plate, a magnetic bead, or a surface plasmon resonance biosensor chip. Methods of attaching antigen-MHC complexes to a solid support are known to the skilled person, and include, for example, using an affinity binding pair, e.g. biotin and streptavidin, or antibodies and antigens. In a preferred embodiment antigen-MHC complexes are labelled with biotin and attached to streptavidin-coated surfaces.

Antigen-MHC complexes of the invention may be in multimeric form, for example, dimeric, or tetrameric, or pentameric, or octomeric, or greater. Examples of suitable methods for the production of multimeric peptide MHC complexes are described in (Greten and Schneck 2002) and references therein. In general, antigen-MHC multimers may be produced using antigen-MHC tagged with a biotin residue and complexed through fluorescent labelled 5 streptavidin. Alternatively, multimeric antigen-MHC complexes may be formed by using immunoglobulin as a molecular scaffold. In this system, the extracellular domains of MHC molecules are fused with the constant region of an immunoglobulin heavy chain separated by a short amino acid linker. Antigen-MHC multimers have also been produced using carrier molecules such as 10 dextran (WO02072631). Multimeric antigen-MHC complexes can be useful for improving the detection of binding moieties, such as T cell receptors, which bind said complex, because of avidity effects.

The antigens of the invention may be presented on the surface of a cell in complex with MHC. Thus, the invention also provides a cell presenting on its surface a complex of the invention. Such a cell may be a mammalian cell, preferably a cell of the immune system, and in particular a specialised antigen presenting cell such as a dendritic cell or a B cell. Other preferred cells include T2 cells (Hosken and Bevan 1990). Cells presenting the antigen or complex of the invention may be isolated, preferably in the form of a population, or provided in a substantially pure form. Said cells may not naturally present the complex of the invention, or alternatively said cells may present the complex at a level higher than they would in nature. Such cells may be obtained by pulsing said cells with the antigen of the invention. Pulsing involves incubating the cells with the antigen for several hours using polypeptide concentrations typically ranging from 10-5 to 10-12 M. Cells may be produced recombinantly. Cells presenting antigen of the invention may be used to isolate T cells and T cell receptors (TCRs) which are activated by, or bind to, said cells, as described in more detail below.

Peptides of the invention may be synthesised using Fmoc chemistry or other standard techniques known to those skilled in the art.

Another convenient way of producing a peptide according to the present invention is to express the nucleic acid encoding it, by use of nucleic acid in an expression system. Such a nucleic acid forms another aspect of the invention.

The skilled person will be able to determine substitutions, deletions and/or additions to such nucleic acids which will still provide a peptide of the present invention. The nucleic acid may be DNA, cDNA, or RNA such as MRNA obtained by cloning or produced by chemical synthesis. For therapeutic use, the nucleic acid is preferably in a form capable of being expressed in the subject to be treated. The peptide of the present invention or the nucleic acid of the present invention may be provided as an isolate, in isolated and/or purified form, or free or substantially free of material with which it is naturally associated. In the case of a nucleic acid, it may be free or substantially free of nucleic acid flanking the gene in the human genome, except possibly one or more regulatory sequence(s) for expression. Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T.

Nucleic acid sequences encoding a peptide of the present invention can be readily prepared by the skilled person, for example using the information and references contained herein and techniques known in the art (for example, see (Sambrook 1989; Ausubel 1992)), given the nucleic acid sequences and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding the polypeptide may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially-available expression system. Another recombinant approach is to amplify the relevant portion of the DNA with suitable PCR primers. Modifications to the sequences can be made, e.g. using site directed mutagenesis, to lead to the expression of modified peptide or to take account of codon preferences in the host cells used to express the nucleic acid.

The present invention also provides constructs in the form of plasmids, vectors, transcription or expression cassettes which comprise at least one nucleic acid as described above. The present invention also provides a recombinant host cell which comprises one or more constructs as above. As mentioned, a nucleic acid encoding a peptide of the invention forms an aspect of the present invention, as does a method of production of the composition which method comprises expression from encoding nucleic acid. Expression may conveniently be achieved by culturing under appropriate conditions recombinant host cells containing the nucleic acid. Following production by expression, a composition may be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, Hela cells, baby hamster kidney cells, NSO mouse melanoma cells and many others. A common, preferred bacterial host is E. coli. The expression of antibodies and antibody fragments in prokaryotic cells such as E. coli is well established in the art. Expression in eukaryotic cells in culture is also available to those skilled in the art as an option for production of a specific binding member, see for recent review, for example (Reff 1993; Trill, Shatzman, and Ganguly 1995). For a review, see for example (Pluckthun 1991). Expression in eukaryotic cells in culture is also available to those skilled in the art as an option for production of a specific binding member, see for recent review, for example (Reff 1993;

### Trill, Shatzman, and Ganguly 1995).

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Molecular Cloning: A Laboratory Manual (Sambrook 1989). Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology (Ausubel 1992).

Thus, a further aspect of the present invention provides a host cell, which may be isolated, containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene.

In one embodiment, the nucleic acid of the invention is integrated into the genome (e.g. chromosome) of the host cell. Integration may be promoted by inclusion of sequences which promote recombination with the genome, in accordance with standard techniques.

The present invention also provides a method which comprises using a construct as stated above in an expression system in order to express a polypeptide as described above. Polypeptides of the invention can be used to identify and/or isolate binding moieties that bind specifically to the polypeptide of the invention. Such binding moieties may be used as immunotherapeutic reagents and may include antibodies. Therefore, in a further aspect, the invention provides a binding moiety that binds the polypeptide of the invention.

Antigens and complexes of the invention can be used to identify and/or isolate binding moieties that bind specifically to the antigen and/or the complex of the invention. Such binding moieties may be used as immunotherapeutic reagents and may include antibodies and TCRs.

In a third aspect, the invention provides a binding moiety that binds the antigen of the invention.

Preferably the binding moiety binds the antigen when said polypeptide is in complex with MHC.

In the latter instance, the binding moiety may bind partially to the MHC, provided that it also binds to the antigen. The binding moiety may bind only the antigen, and that binding may be specific. The binding moiety may bind only the antigen-MHC complex and that binding may be specific.

When used with reference to binding moieties that bind the complex of the invention, “specific” is generally used herein to refer to the situation in which the binding moiety does not show any significant binding to one or more alternative antigen-MHC complexes other than the antigen-MHC complex of the invention.

The binding moiety may be a T cell receptor (TCR). TCRs are described using the International Immunogenetics (IMGT) TCR nomenclature, and links to the IMGT public database of TCR sequences. The unique sequences defined by the IMGT nomenclature are widely known and accessible to those working in the TCR field. For example, they can be found in the “T cell Receptor Factsbook”, (2001) LeFranc and LeFranc, Academic Press, ISBN 0-12-441352-8; Lefranc, (2011), Cold Spring Harb Protoc 2011 (6): 595-603; Lefranc, (2001), Curr Protoc Immunol Appendix 1: Appendix 10; (Lefranc 2003), and on the IMGT website (www.IMGT.org). Briefly, alpha beta TCRs consist of two disulphide linked chains. Each chain (alpha and beta) is generally regarded as having two domains, namely a variable and a constant domain. A short joining region connects the variable and constant domains and is typically considered part of the alpha variable region. Additionally, the beta chain usually contains a short diversity region next to the joining region, which is also typically considered part of the beta variable region.

The TCRs may be in any format known to those in the art. For example, the TCRs may be αβ heterodimers, or they may be in single chain format (such as those described in WO9918129).

Single chain TCRs include αβ TCR polypeptides of the type: Vα-L-Vβ, Vβ-L-Vα, Vα-Cα-L-Vβ, Vα-L-Vβ-Cβ or Vα-Cα-L-Vβ-Cβ, optionally in the reverse orientation, wherein Vα and Vβ are TCR α and β variable regions respectively, Cα and Cβ are TCR α and β constant regions respectively, and L is a linker sequence. The TCR may be in a soluble form (i.e. having no transmembrane or cytoplasmic domains); or may contain full length alpha and beta chains.

The TCR may be provided on the surface of a cell, such as a T cell. The cell may be a mammalian cell, such as a human cell.

The cell may be used in medicine, in particular for treating cancer. The cancer may be a solid tumour or a haematological neoplasia. The cancer may be thyroid, colorectal, urothelial, stomach, liver, carcinoid, pancreatic, renal, prostate, lung, breast and gynaecological cancers. The cells may be autologous to the subject to be treated or not autologous to the subject to be treated.

The alpha and/or beta chain constant domain of the TCR may be truncated relative to the native/naturally occurring TRAC/TRBC sequences. In addition, the TRAC/TRBC may contain modifications. For example, the alpha chain extracellular sequence may include a modification relative to the native/naturally occurring TRAC whereby amino acid T48 of TRAC, with reference to IMGT numbering, is replaced with C48. Likewise, the beta chain extracellular sequence may include a modification relative to the native/naturally occurring TRBC1 or TRBC2 whereby S57 of TRBC1 or TRBC2, with reference to IMGT numbering, is replaced with C57. These cysteine substitutions relative to the native alpha and beta chain extracellular sequences enable the formation of a non-native interchain disulphide bond which stabilises the refolded soluble TCR, i.e. the TCR formed by refolding extracellular alpha and beta chains (WO 03/020763). This non-native disulphide bond facilitates the display of correctly folded TCRs on phage (Li et al. 2005). In addition, the use of the stable disulphide linked soluble TCR enables more convenient assessment of binding affinity and binding half-life. Alternative positions for the formation of a non-native disulphide bond are described in WO 03/020763.

The variable domain of each chain is located N-terminally and comprises three Complementarity Determining Regions (CDRs) embedded in a framework sequence (FR). The CDRs comprise the recognition site for peptide-MHC binding. There are several genes coding for alpha chain variable (Vα) regions and several genes coding for beta chain variable (Vβ) regions, which are distinguished by their framework, CDR1 and CDR2 sequences, and by a partly defined CDR3 sequence. The Vα and VB genes are referred to in IMGT nomenclature by the prefix TRAV and TRBV respectively (Folch et al. 2000; Lefranc 2001) “T cell Receptor Factsbook”, Academic Press). Likewise there are several joining or J genes, termed TRAJ or TRBJ, for the alpha and beta chain respectively, and for the beta chain, a diversity or D gene termed TRBD (Folch et al. 2000; Lefranc 2001) “T cell Receptor Factsbook”, Academic Press). The huge diversity of T cell receptor chains results from combinatorial rearrangements between the various V, J and D genes, which include allelic variants, and junctional diversity (Arstila et al. 1999) (Robins et al. 2009). The constant, or C, regions of TCR alpha and beta chains are referred to as TRAC and TRBC respectively (Lefranc, (2001), Curr Protoc Immunol Appendix 1: Appendix 10). TCRs of the invention may be engineered to include mutations. Methods for producing mutated high affinity TCR variants such as phage display and site directed mutagenesis and are known to those in the art (for example see WO 04/044004 and Li et al. (Li et al. 2005).

TCRs may also be may be labelled with an imaging compound, for example a label that is suitable for diagnostic purposes. Such labelled high affinity TCRs are useful in a method for detecting a TCR ligand selected from CD1-antigen complexes, bacterial superantigens, and MHC-peptide/superantigen complexes, which method comprises contacting the TCR ligand with a high affinity TCR (or a multimeric high affinity TCR complex) which is specific for the TCR ligand; and detecting binding to the TCR ligand. In multimeric high affinity TCR complexes such as those described in Zhu et al., (Zhu et al. 2006), (formed, for example, using biotinylated heterodimers) fluorescent streptavidin (commercially available) can be used to provide a detectable label. A fluorescently labelled multimer is suitable for use in FACS analysis, for example to detect antigen presenting cells carrying the peptide for which the high affinity TCR is specific.

According to the invention, Cytokeratin 8 peptides containing citrulline can be used as targets for cancer immunotherapy via T cell receptors (TCRs). TCRs are designed to recognise short peptide antigens that are displayed on the surface of APCs in complex with MHC molecules (Davis et al. 1998). The identification of particular citrulline containing peptides is advantageous for the development of novel immunotherapies. Such therapeutic TCRs may be used, for example, as soluble targeting agents for the purpose of delivering cytotoxic or immune effector agents to the tumour (Boulter et al. 2003; Liddy et al. 2012; McCormack et al. 2013), or alternatively they may be used to engineer T cells for adoptive therapy (June et al. 2014).

A TCR of the present invention (or multivalent complex thereof) may alternatively or additionally be associated with (e.g. covalently or otherwise linked to) a therapeutic agent which may be, for example, a toxic moiety for use in cell killing, or an immunostimulating agent such as an interleukin or a cytokine. A multivalent high affinity TCR complex of the present invention may have enhanced binding capability for a TCR ligand compared to a nonmultimeric wild-type or high affinity T cell receptor heterodimer. Thus, the multivalent high affinity TCR complexes according to the invention are particularly useful for tracking or targeting cells presenting particular antigens in vitro or in vivo, and are also useful as intermediates for the production of further multivalent high affinity TCR complexes having such uses. The high affinity TCR or multivalent high affinity TCR complex may therefore be provided in a pharmaceutically acceptable formulation for use in vivo.

TCRs of the invention may be used as therapeutic reagents. In this case the TCRs may be in soluble form and may preferably be fused to an immune effector. Suitable immune effectors include but are not limited to, cytokines, such as IL-2 and IFN-α; superantigens and mutants thereof; chemokines such as IL-8, platelet factor 4, melanoma growth stimulatory protein; antibodies, including fragments, derivatives and variants thereof, that bind to antigens on immune cells such as T cells or NK cell (e.g. anti-CD3, anti-CD28 or anti-CD16); and complement activators.

The binding moiety of the invention may be an antibody. The term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term “antibody” includes antibody fragments, derivatives, functional equivalents and homologues of antibodies, humanised antibodies, including any polypeptide comprising an immunoglobulin binding domain, whether natural or wholly or partially synthetic and any polypeptide or protein having a binding domain which is, or is homologous to, an antibody binding domain. Chimeric molecules comprising an immunoglobulin binding domain, or equivalent, fused to another polypeptide are therefore included. Cloning and expression of chimeric antibodies are described in EP-A-0120694 and EP-A-0125023. A humanised antibody may be a modified antibody having the variable regions of a non-human, e.g. murine, antibody and the constant region of a human antibody. Methods for making humanised antibodies are described in, for example, U.S. Pat. No. 5,225,539. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to herein as “mab”.

It is possible to take an antibody, for example a monoclonal antibody, and use recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin (see, for instance, EP-A-184187, GB 2188638A or EP-A-239400). A hybridoma (or other cell that produces antibodies) may be subject to genetic mutation or other changes, which may or may not alter the binding specificity of antibodies produced.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al. 1989) which consists of a VH domain; (v) isolated CDR regions; (vi) F(ab’)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. 1988; Huston et al. 1988); (viii) bispecific single chain Fv dimers (PCT/US92/09965) and (ix) “diabodies”, multivalent or multispecific fragments constructed by gene fusion (WO94/13804; (Holliger and Winter 1993)). Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804). Where bispecific antibodies are to be used, these may be conventional bispecific antibodies, which can be manufactured in a variety of ways (Holliger and Winter 1993), e.g. prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins” described in (Traunecker, Lanzavecchia, and Karjalainen 1991). Bispecific diabodies, as opposed to bispecific whole antibodies, may also be useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied, and an antibody of appropriate specificity selected. An “antigen binding domain” is the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains. An antigen binding domain may comprise an antibody light chain variable region (VL) and an antibody heavy chain variable region (VH).

Also encompassed within the present invention are binding moieties based on engineered protein scaffolds. Protein scaffolds are derived from stable, soluble, natural protein structures which have been modified to provide a binding site for a target molecule of interest. Examples of engineered protein scaffolds include, but are not limited to, affibodies, which are based on the Z-domain of staphylococcal protein A that provides a binding interface on two of its a-helices (Nygren 2008); anticalins, derived from lipocalins, that incorporate binding sites for small ligands at the open end of a beta-barrel fold (Skerra 2008), nanobodies, and DARPins. Engineered protein scaffolds are typically targeted to bind the same antigenic proteins as antibodies, and are potential therapeutic agents. They may act as inhibitors or antagonists, or as delivery vehicles to target molecules, such as toxins, to a specific tissue in vivo (Gebauer and Skerra 2009). Short peptides may also be used to bind a target protein. Phylomers are natural structured peptides derived from bacterial genomes. Such peptides represent a diverse array of protein structural folds and can be used to inhibit/disrupt protein-protein interactions in vivo (Watt 2006).

As discussed, the inventors have found that certain modified Cyk8 antigens are associated with tumours and citrullinated peptides stimulate T cell responses which can be used to raise an immune response against tumours. The present invention provides an antigen of the first aspect, a complex of the second aspect, and/or a binding moiety of the third aspect for use in medicine. The antigen of the first aspect, complex of the second aspect, and/or binding moiety of the third aspect can be used in a method for treating cancer. Also provided are the use of an antigen of the first aspect, a complex of the second aspect, and/or a binding moiety of the third aspect in the manufacture of a medicament for the treatment of cancer, as well as a method of treating cancer, comprising administering an antigen of the first aspect, a complex of the second aspect, and/or a binding moiety of the third aspect of the invention to a subject in need of such treatment. Antigens in accordance with the present invention may be used alone or in combination as a pool. In addition, they may be used in combination with other therapeutic agents, such as anti-cancer agents including but not limited to checkpoint blockade drugs such as ipilimumab, pembrolizumab and nivolumab.

The inventors are the first to show that citrullinated Cytokeratin 8 peptides can stimulate potent T cell responses. The invention provides suitable means for local stimulation of an immune response directed against tumour tissue in a subject. T cells specific for these Cyk8 cit peptides could target tumour cells to elicit strong anti-tumour effects in vivo, thus providing the first evidence for the use of Cyk8 cit epitopes as vaccine targets for cancer therapy. Stimulation of an immune response directed against a vaccine target includes the natural immune response of the patient and immunotherapeutic treatments aiming to direct the immune response against the tumour (e.g. checkpoint inhibitors, CAR-Ts against tumour antigens and other tumour immunotherapies). Such support or induction of the immune response may in various clinical settings be beneficial in order to initiate and maintain the immune response and evade the tumour-mediated immunosuppression that often blocks this activation. These responses may be tolerised for the treatment of autoimmune diseases.

In some embodiments, the cellular immune response is specific for the stress induced post translationally modified peptide wherein immune response includes activation of T cells expressing TCRαβ or γδ. The present invention also relates to TCRs, individual TCR subunits (alone or in combination), and subdomains thereof, soluble TCRs (sTCRs), for example, soluble αβ dimeric TCRs having at least one disulphide inter-chain bond between constant domain residues that are not present in native TCRs, and cloned TCRs, said TCRs engineered into autologous or allogeneic T cells or T cell progenitor cells, and methods for making same, as well as other cells bearing said TCR.

The cancer may be thyroid, colorectal, urothelial, stomach, liver, carcinoid, pancreatic, renal, prostate, lung, breast and gynaecological cancer.

The present invention provides a pharmaceutical composition comprising an antigen, complex and/or binding moiety of the present invention. The formulation may be formulated with an adjuvant or other pharmaceutically acceptable vaccine component. In particular embodiments, the adjuvant is a TLR ligand such as CpG (TLR9) MPLA (TLR4), imiquimod (TLR7), poly I: C (TLR3) or amplivant TLR1/2 ligand, GMCSF, an oil emulsion, a bacterial product or whole inactivated bacteria.

The antigen may be a T or B cell antigen. Peptides in accordance with the present invention may be used alone or in combination. In addition, they may be used in combination with other therapeutic agents, such as anti-cancer agents including but not limited to checkpoint blockade drugs such as ipilimumab.

Antigens in accordance with the invention may be delivered in vivo as a peptide, optionally in the form of a peptide as disclosed in WO02/058728. The inventors have surprisingly found that antigens of the invention give rise to strong immune responses when administered as a peptide. Such peptides may be administered as just the sequence of the peptide, or as a polypeptide containing the antigen, or even as the full-length protein. Alternatively, antigens in accordance with the invention may be administered in vivo as a nucleic acid encoding the antigen, encoding a polypeptide containing the antigen or even encoding the full-length protein. Such nucleic acids may be in the form of a mini gene, i.e. encoding a leader sequence and the antigen or a leader sequence and full-length protein.

As used herein, the term “treatment” includes any regime that can benefit a human or non-human animal. The antigen and/or nucleic acid and/or complex and/or binding moiety may be employed in combination with a pharmaceutically acceptable carrier or carriers to form a pharmaceutical composition. Such carriers may include, but are not limited to, saline, buffered saline, dextrose, liposomes, water, glycerol, ethanol and combinations thereof.

It is envisaged that injections will be the primary route for therapeutic administration of the compositions of the invention although delivery through a catheter or other surgical tubing may also be used. Some suitable routes of administration include intravenous, subcutaneous, intradermal, intraperitoneal and intramuscular administration. Liquid formulations may be utilised after reconstitution from powder formulations.

For intravenous injection, or injection at the site of affliction, the active ingredient will be in the form of a parentally acceptable aqueous solution which is pyrogen-free, has suitable pH, is isotonic and maintains stability. Those of relevant skill in the art are well able to prepare suitable solutions using, for example, isotonic vehicles such as sodium chloride injection, Ringer's Injection or Lactated Ringer's Injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. Where the formulation is a liquid it may be, for example, a physiologic salt solution containing non-phosphate buffer at pH 6.8-7.6, or a lyophilised powder.

The composition may be administered in a localised manner to a tumour site or other desired site or may be delivered in a manner in which it targets tumour or other cells. In some embodiments, the antigen are administered without an adjuvant for a cellular immune response including activation of T cells expressing TCRαβ or γδ.

The compositions are preferably administered to an individual in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. The compositions of the invention are particularly relevant to the treatment of cancer, and in the prevention of the recurrence of such conditions after initial treatment or surgery. Examples of the techniques and protocols mentioned above can be found in Remington's Pharmaceutical Sciences (Remington 1980). A composition may be administered alone or in combination with other treatments, either simultaneously or sequentially dependent upon the condition to be treated. Other cancer treatments include other monoclonal antibodies, other chemotherapeutic agents, other radiotherapy techniques or other immunotherapy known in the art. One particular application of the compositions of the invention is as an adjunct to surgery, i.e. to help to reduce the risk of cancer reoccurring after a tumour is removed. The compositions of the present invention may be generated wholly or partly by chemical synthesis. The composition can be readily prepared according to well-established, standard liquid or, preferably, solid-phase peptide synthesis methods, general descriptions of which are broadly available (see, for example, in Solid Phase Peptide Synthesis, 2nd edition (Stewart 1984), in The Practice of Peptide Synthesis (Bodanzsky 1984) and Applied Biosystems 430A User's Manual, ABI Inc., or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

The antigens, complexes, nucleic acid molecules, vectors, cells and binding moieties of the invention may be non-naturally occurring and/or purified and/or engineered and/or recombinant and/or isolated and/or synthetic.

The invention also provides a method of identifying a binding moiety that binds a complex of the invention, the method comprising contacting a candidate binding moiety with the complex and determining whether the candidate binding moiety binds the complex.

Preferred features of each aspect of the invention are as for each of the other aspects mutatis mutandis. The prior art documents mentioned herein are incorporated to the fullest extent permitted by law.

### EXAMPLES

The present invention will now be described further with reference to the following examples and the accompanying drawings.

FIG. 1: Isoforms of Cytokeratin 8 and alignment of human Cytokeratin 8 with equivalent sequences from other species

Two different isoforms of Cytokeratin 8 exist (Isoform1 and isoform 2, FIG. 1A). Alignment of human Cytokeratin 8 (B) with equivalent sequences from other species (Mouse, Rat, Bovine, Pig, Horse, Chicken, Felis, Dog, Rabbit and sheep).

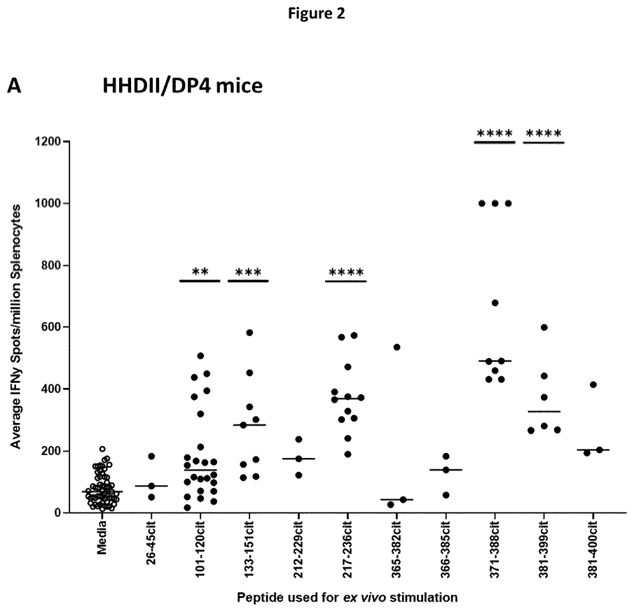

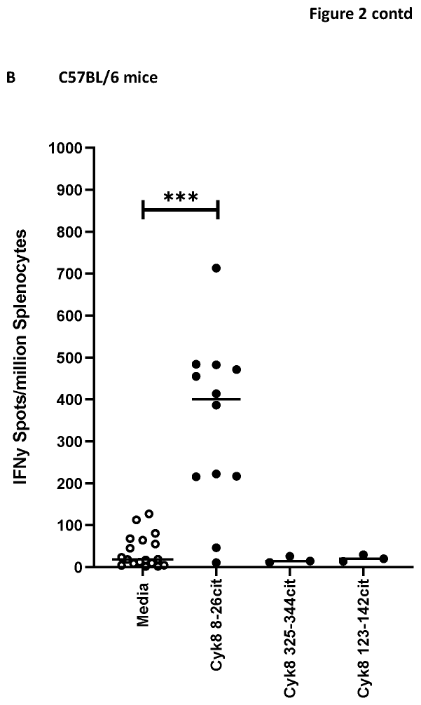

FIG. 2: Screening IFNγ responses to citrullinated Cytokeratin 8 peptide pools

Transgenic mouse strains expressing HHDII/DP4 and parental C57BL mice were used to screen for IFNγ responses to peptide (A and B respectively). Mice were immunised with peptide pools of up to 2-3 non-overlapping human Cyk8 citrullinated peptides over three weeks. Splenocytes were harvested 21 days after the initial dose was administered. Ex vivo responses to stimulation with human Cyk8 cit peptides was assessed by IFNγ ELISpot. Media only responses were used as a negative control. n≥3 symbol represents mean response for individual mice, line represents median value between mice. Statistical significance of peptide response was compared to media only responses for each pool and determined using ANOVA with Dunnett's post-hoc test * p<0.5, ** p<0.01, *** p<0.001, **** p<0.0001, only significant p values are shown. Peptide titrations were performed using Cyk8 133-151 cit, Cyk8 217-236 cit, Cyk8 371-388 cit peptides (C) at concentrations ranging from 0.001 μg/ml to 10 μg/ml, data was plotted on a log scale and EC50 values calculated using GraphPad Prism. Only curves are shown where the curve fit (R squared) was above 0.95.

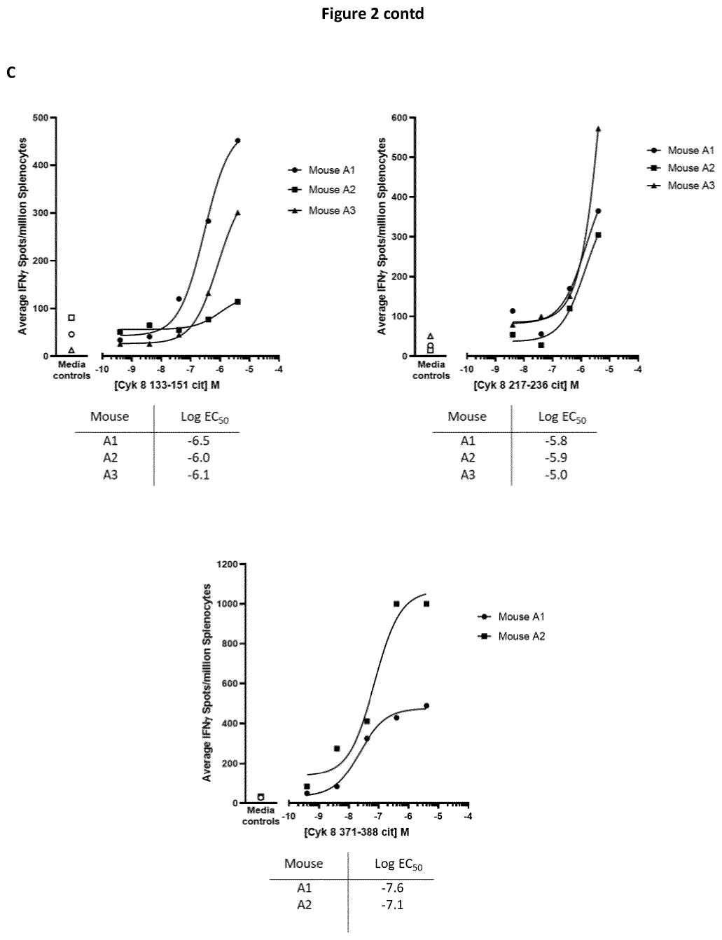

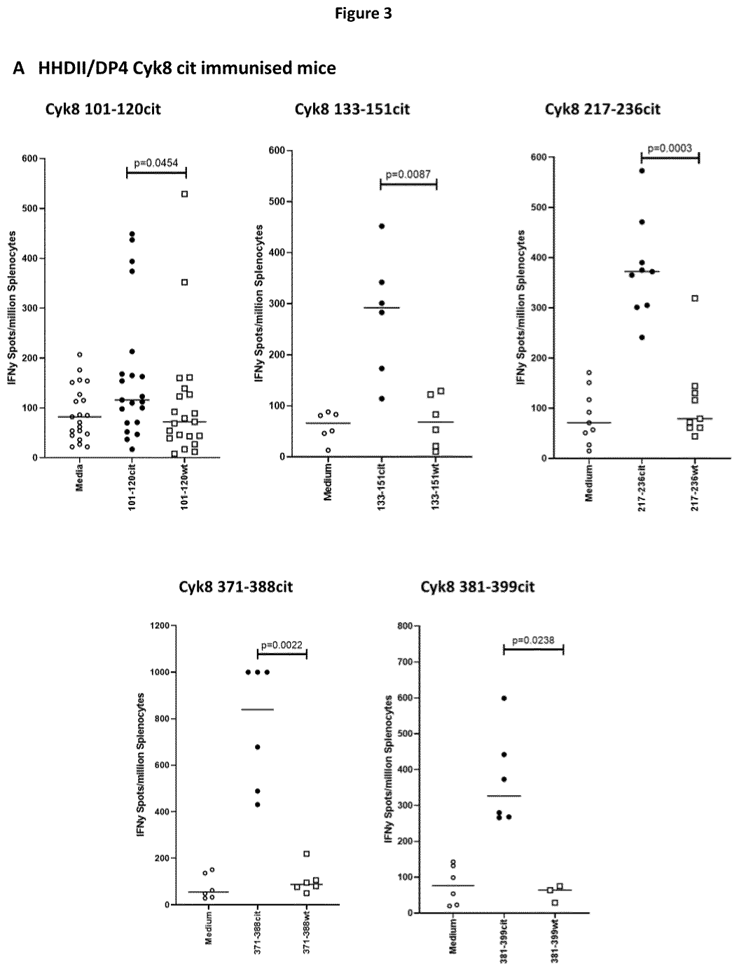

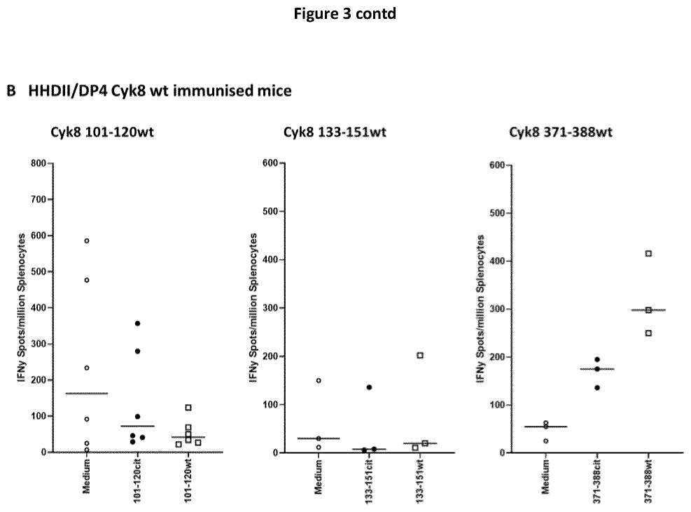

FIG. 3: Multiple citrullinated Cytokeratin 8 peptides induce strong IFNγ responses

HLA-DP4 mice were immunised with three doses of Cyk8 101-120, Cyk8 133-151, Cyk8 217-236, Cyk8 371-388 and Cyk8 381-399 citrullinated peptides (A) or 101-120, 133-151 and 371-388 wild type peptides (B) over a course of three weeks, with weekly immunisations. Splenocytes were collected 7 days after the third dose was administered. Ex vivo IFNγ ELISpot was performed to determine the response to Cyk8 citrullinated and wildtype peptides. n≥3 symbol represents mean response for individual mice, line represents median value between mice. Statistical significance of peptide responses to Cyk8 cit was compared to the response to Cyk8 wt peptides using Mann-Whitney test, only significant p values are shown.

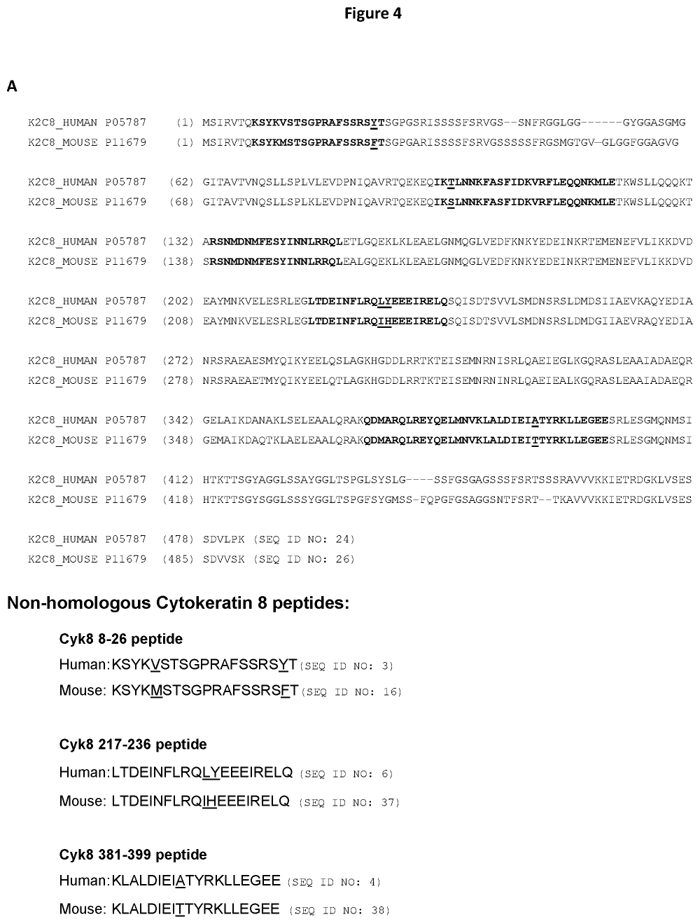

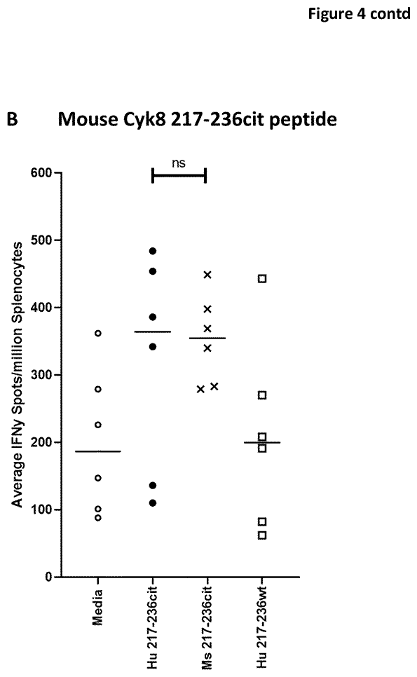

FIG. 4: Homologous human and murine citrullinated Cytokeratin 8 217-236 peptide induces similar IFNγ responses

Sequence comparison of murine Cytokeratin 8 and human Cytokeratin 8 (A). Mouse and human Cyk8 217-236 cit was tested in HHDII/DP4 mice (B). Mice were immunised with three doses of mouse Cyk8 217-236 citrullinated peptide over a course of three weeks, with weekly immunisations. Splenocytes were collected 7 days after the third dose was administered. Ex vivo IFNγ ELISpot was performed to determine the response to murine and human Cyk8 217-236 citrullinated and human Cyk8 217-236 wildtype peptide (B). n≥3 symbol represents mean response for individual mice, line represents median value between mice. Statistical significance of peptide responses to mouse Cyk8 217-236 cit, human Cyk8 217-236 cit and human Cyk8 wt peptides were compared using ANOVA with Dunnett's multiple comparison test * p<0.5, ** p<0.01, *** p<0.001, **** p<0.0001, ns=not significant.

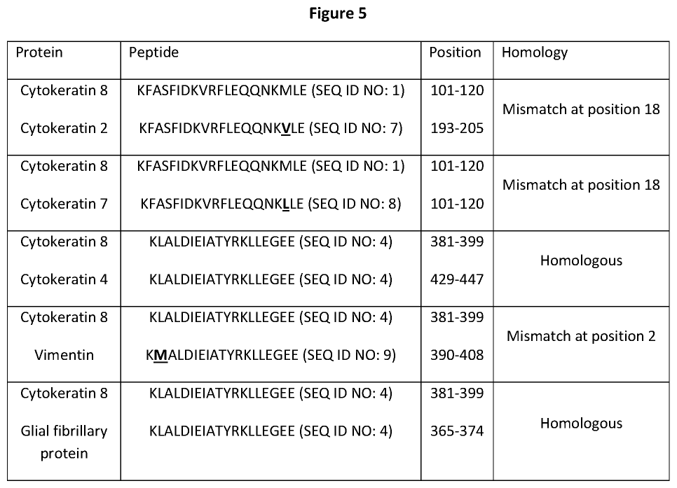

FIG. 5: Cytokeratin 8 peptides share homology with other Cytokeratin's and

Due to the high degree of sequence homology between the different Cytokeratin's a peptide blast search was performed on the Cytokeratin 8 peptides that were identified here. The results are shown for peptides that are homologous or have a 1 amino acid mismatch.

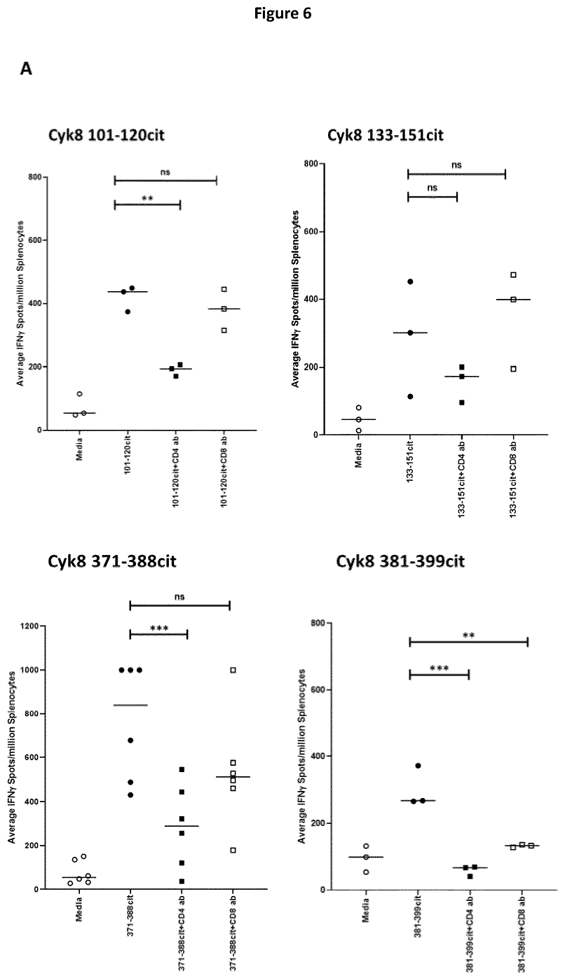

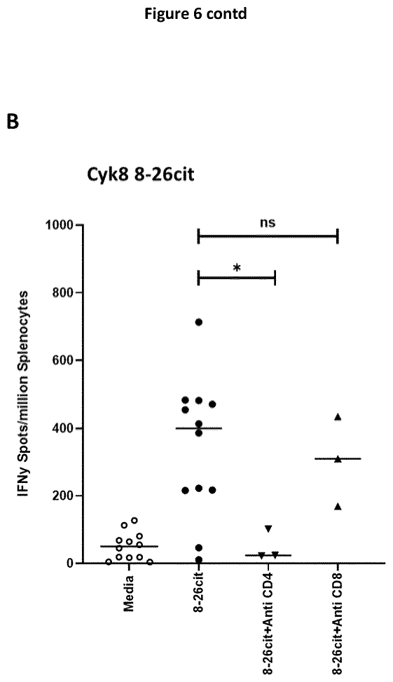

FIG. 6: Characterisation of Cytokeratin 8 (Cyk8) Cit peptide responses in HHDII/DP4 and C57BL/6 mice

HHDII/DP4 (A) and C57BL/6 (B) mice were immunised with three doses of individual citrullinated peptide over a course of three weeks, with weekly immunisations. Splenocytes were collected 7 days after the third dose was administered. Ex vivo ELISpots were performed on day 21. Splenocytes were re-stimulated with media or cit peptide in the presence of CD4/CD8 blocking antibodies. n≥3 symbol represents mean response for individual mice, line represents median value between mice. Statistical significance of responses in the presence of CD4/CD8 blocking antibodies was compared to the responses in the absence of blocking antibodies using using ANOVA with Dunnett's multiple comparison test * p<0.5, ** p<0.01, *** p<0.001, **** p<0.0001, ns=not significant.

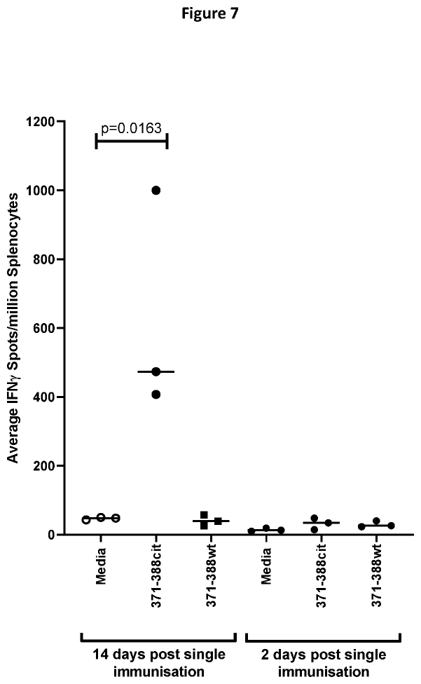

FIG. 7: Responses develop in mice 14 days after a single immunisation suggestion these are naive responses

HHDII/DP4 transgenic mice were immunised with a single dose of Cytokeratin 8 371-388 cit peptide in CpG/MPLA 2 or 14 days before mice were sacrificed and ex vivo ELISpots were performed to determine the IFNγ responses. n≥3 symbol represents mean response for individual mice, line represents median value between mice. p values represent significant difference compared to peptide responses at day 14, only significant p values are shown.

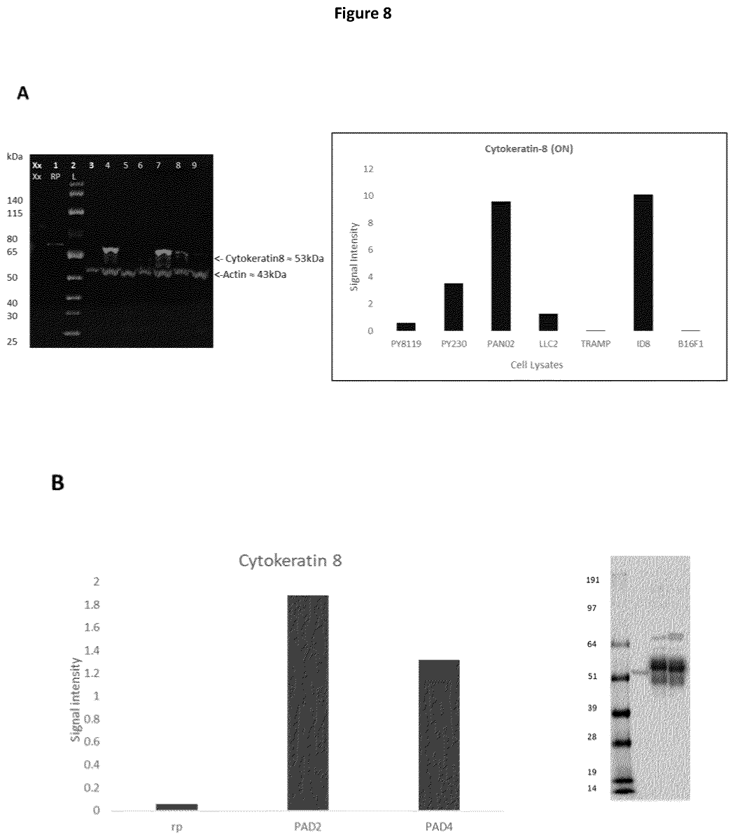

FIG. 8: Expression of Cytokeratin 8 on cancer cell lines and detection of citrullinated Cytokeratin 8 following in vitro citrullination Immunoblot (A) of lysates from cancer cell lines, recombinant Cytokeratin 8 (Lane 1), ladder (Lane 2), PY230 (Lane 3), PANO2 (Lane 4), LLC2 (Lane 5), TRAMP (Lane 6), ID8 (Lane 7),

B16F1 (Lane 8), PY8119 (Lane 9) probed for Cytokeratin 8 and β actin. The bands correspond to the expected size for Cytokeratin 8 (53 kDa) and β-actin (43 kDa). In vitro citrullination of Cyk8 was performed in the presence of either PAD2 or PAD4 (B), recombinant Cytokeratin 8 (Lane 1), Cytokeratin 8+PAD2 (Lane 2), Cytokeratin 8+PAD4 (Lane 3).

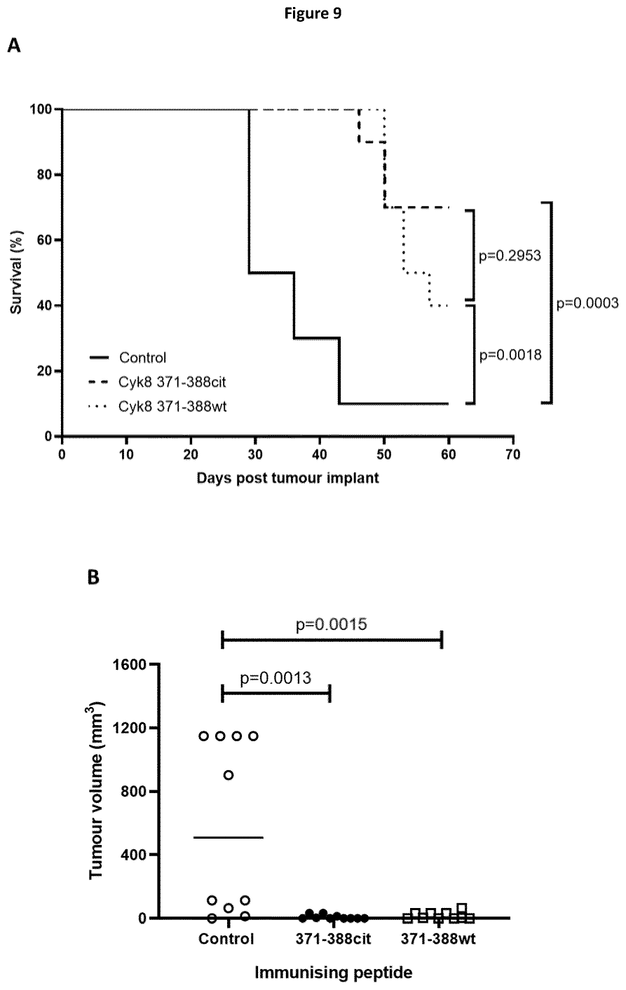

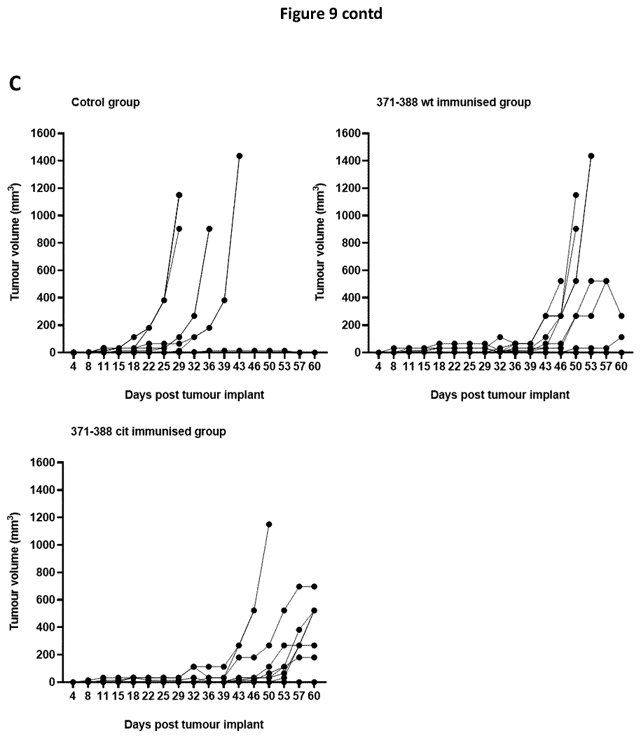

FIG. 9: Human Cytokeratin 8 371-388 cit peptide provides an in vivo survival advantage in anti-tumour studies

HHDII/DP4 mice were challenged with B16 tumour with IFNγ inducible DP4. Four days later the HHDII/DP4 mice were immunised with Cyk8 371-388 cit or Cyk8 371-388 wt peptide on days 4, 11 and 18. Overall survival (A), tumour volume (B) at day 29 and tumour volume throughout the study (C), post tumour implant are shown for unimmunised control mice and mice immunised with either Cyk8 371-388 cit peptide or Cyk8 371-388 wt peptide. Statistical differences between immunised and control mice were determined by Mantel-Cox test, p values are shown. The tumour volume medians and p values are shown as determined by Mann Whitney U test, n=10 in all group.

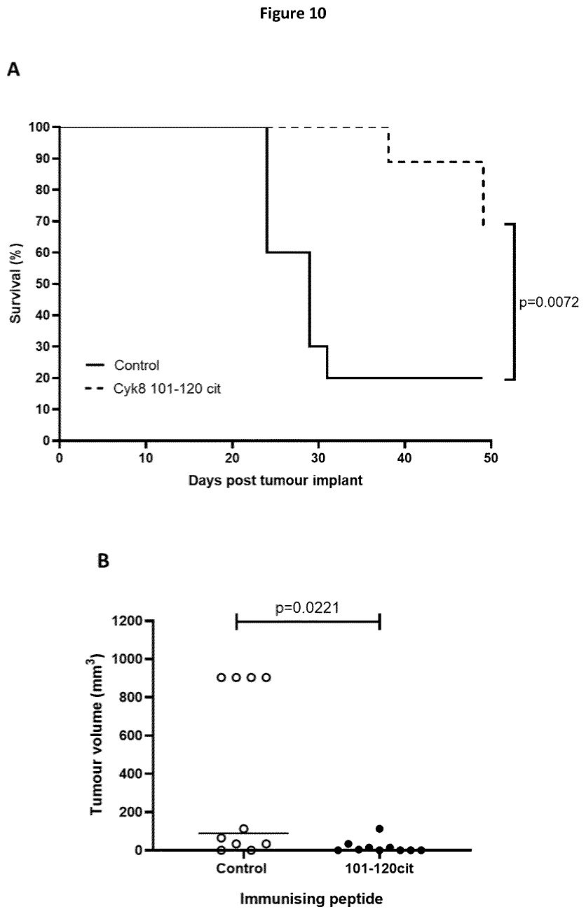

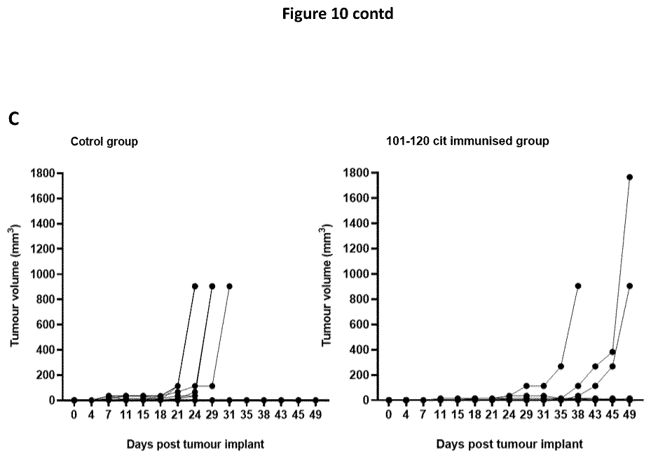

FIG. 10: Human Cytokeratin 8 101-120 cit peptide provides an in vivo survival advantage in anti-tumour studies

HHDII/DP4 mice were challenged with B16 tumour with IFNγ inducible DP4. Four days later the HHDII/DP4 mice were immunised with Cyk8 371-388 cit peptide on days 4, 11 and 18. Overall survival (A), tumour volume (B) at day 24 and tumour volume throughout the study (C), post tumour implant are shown for unimmunised control mice and mice immunised with Cyk8 101-120 cit peptide. Statistical differences between immunised and control mice were determined by Mantel-Cox test, p values are shown. The tumour volume medians and p values are shown as determined by Mann Whitney U test, n=10 in all group.

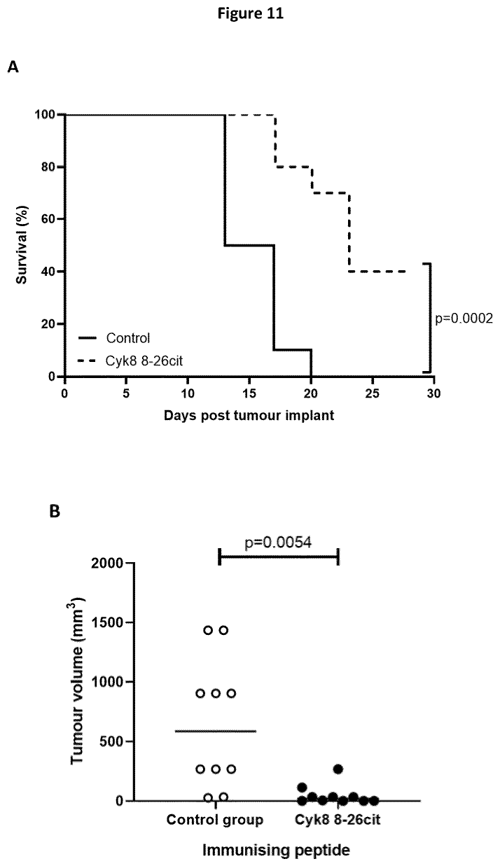

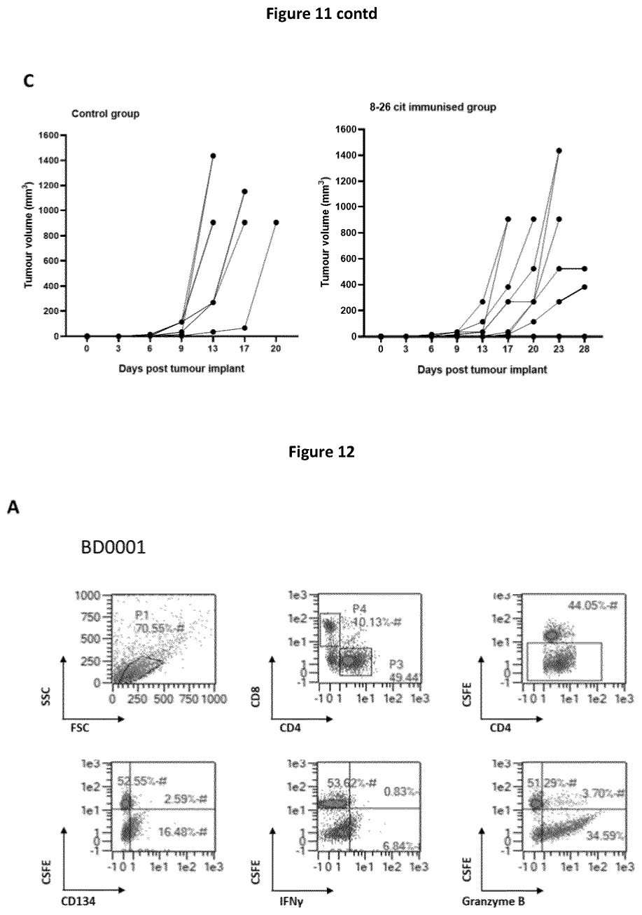

FIG. 11: Cytokeratin 8 8-26 cit peptide provides an in vivo survival advantage in anti-tumour studies

C57BL/6 were challenged with B16 tumour (parental non-transfected cell line). Four days later the C57BL/6 mice were immunised with Cyk8 8-26 cit peptide. Overall survival (A), tumour volume (B) at day 13 and tumour volume throughout the study (C), post tumour implant are shown for unimmunised control mice and mice immunised with Cyk8 8-26 cit peptide. Statistical differences between immunised and control mice were determined by Mantel-Cox test, p values are shown. The tumour volume medians and p values are shown as determined by Mann Whitney U test, n=10 in all group.

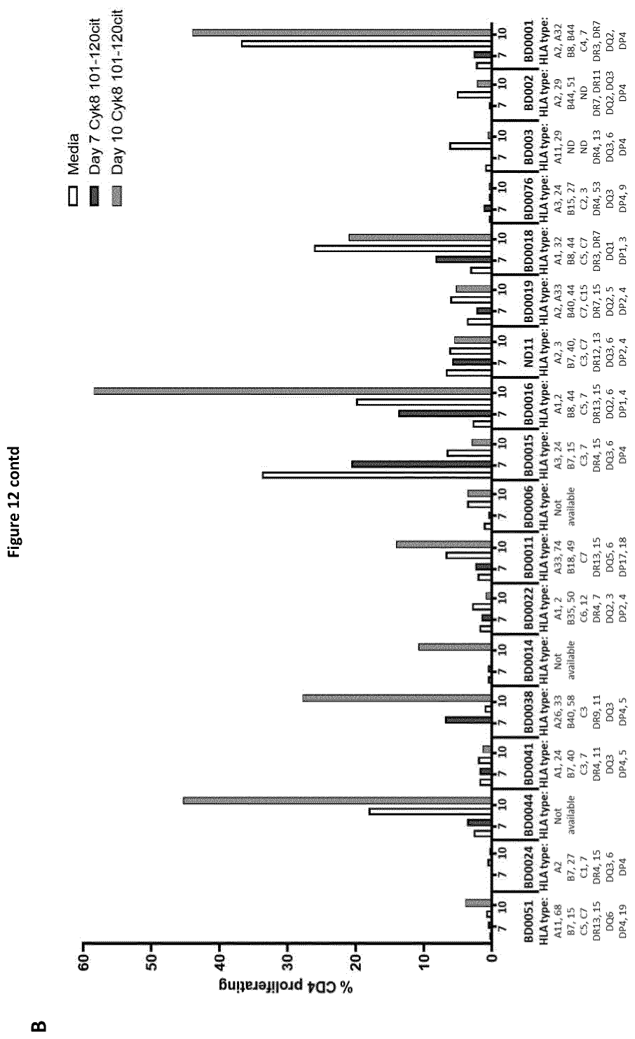

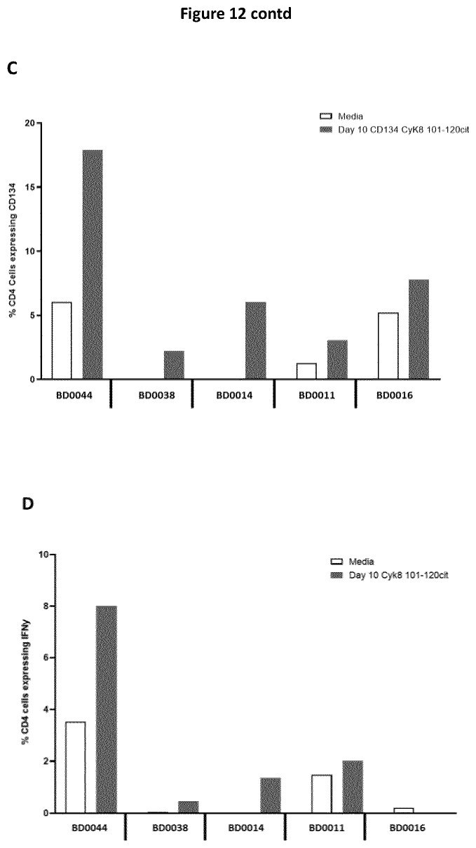

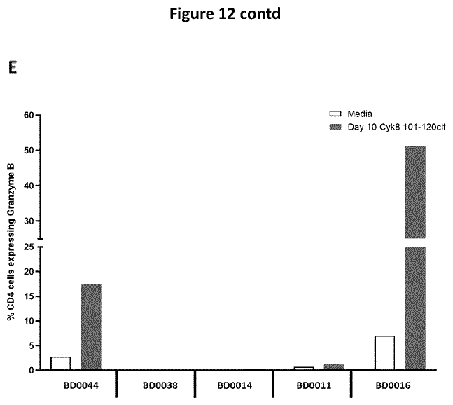

FIG. 12: Human Cytokeratin 101-120 cit peptide induces responses in PBMCs from healthy donors

PBMCs were isolated from 18 healthy donors, HLA typing was performed on the majority of donors, 13 HLA-DP4 positive donors, 2 HLA-DP4 negative donors and for 3 donors the HLA type was not determined. PBMCs isolated from each donor was cultured with media or human Cyk8 101-120 cit peptide. PMBCs were labelled with CSFE prior to stimulation with Cyk8 101-120 cit peptide, a representative flow cytometry plot is shown (A). The proliferative responses of CD4 T cell populations within the CSFE labelled cell population was assessed by flow cytometry on days 7 and 10 (B). The expression of CD134 (C), IFNγ (D) and Granzyme B (E) on proliferating CD4 T cells was assessed on days 7 and 10, responses on day 10 are represented.

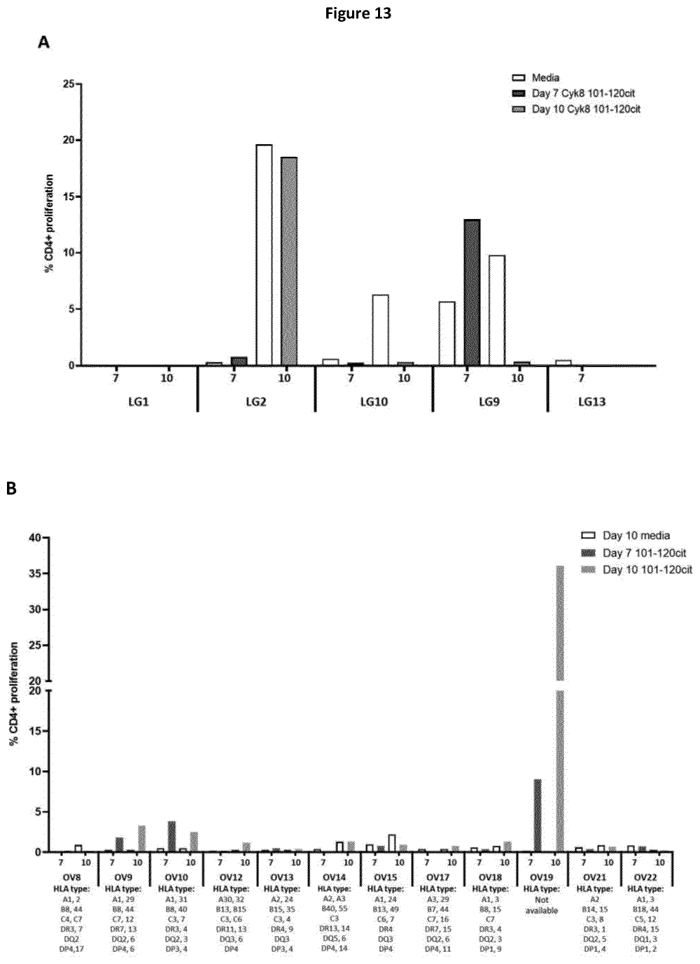

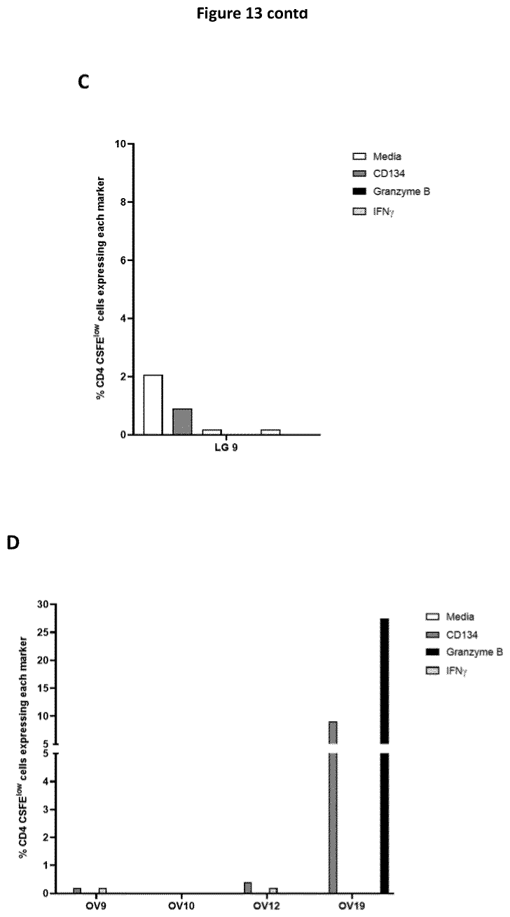

FIG. 13: Human Cytokeratin 101-120 cit peptide induces responses in PBMCs from cancer patients

PBMCs were isolated from 5 lung cancer patients and 12 ovarian cancer patients, HLA typing was not performed on the lung cancer patients but performed for the majority of ovarian cancer patients, 9 HLA DP4 positive ovarian cancer patients, 2 HLA-DP4 negative ovarian cancer patients, HLA types was not performed for 1 ovarian cancer patient. PBMCs isolated from each donor was cultured with media or human Cyk8 101-120 cit peptide. PMBCs were labelled with CSFE prior to stimulation with Cyk8 101-120 cit peptide. The proliferative responses of CD4 T cell populations within the CSFE labelled cell population was assessed by flow cytometry on days 7 and 10 in PBMCs from lung cancer patients (A) and ovarian cancer patients (B). The expression of CD134, IFNγ and Granzyme B on proliferating CD4 T cells was assessed on days 7 and 10, responses on day 10 are represented for the lung cancer patient (C) and ovarian cancer patients (D).

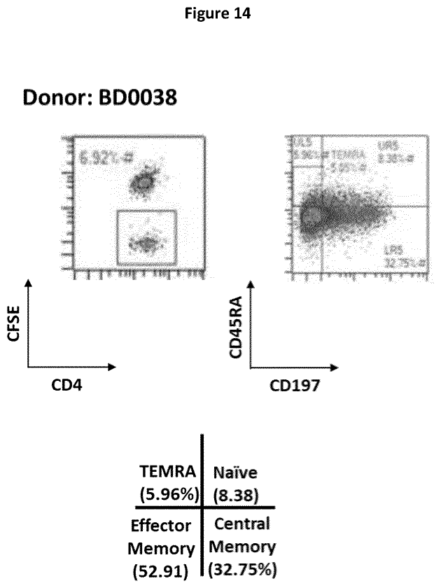

FIG. 14: Immune response to human Cytokeratin 8 101-120 cit peptide is mediated by memory T cells

CSFE^{low} T cells specific for Cyk8 101-120 cit peptide were phenotyped using markers to identify memory and naive T cell populations, the phenotype was assessed by flow cytometry on day 10.

### METHODS

1.1. Commercial mAbs

Anti-IFNγ antibody (clone XMG1.2), anti-mouse CD4 (clone GK1.5), anti-mouse CD8 (clone 2.43) and anti-human CD4 (clone OKT-4) were purchased from BioXcell, USA. Anti-human CD134 (clone REA621) and anti-human CD8 (clone REA734) were purchased from Miltenyi, Germany. Anti-human CD4 (clone RPA-T4), anti-human Granzyme B (clone GB11) were purchased from Thermo Fisher Scientific, USA, anti-human IFNγ (clone E780) was purchased from eBioscience, USA.

### 1.2. Cell Lines

The murine melanoma B16F1, murine pancreatic pan02 cell lines were obtained from the American Tissue Culture Collection (ATCC) and cultured in RPMI medium 1640 (GIBCO/BRL) supplemented with 10% fetal calf serum (FCS), L-glutamine (2 mM) and sodium bicarbonate buffered unless otherwise stated. The murine transgenic TRAMP cell was obtained from ATCC and cultured in dulbecco's modified Eagle's medium with 4 mM L-glutamine adjusted to contain 1.5 g/L sodium bicarbonate and 4.5 g/L glucose supplemented with 0.005 mg/ml bovine insulin and 10 nM dehydroisoandrosterone, 90%; fetal bovine serum, 5%; Nu-Serum IV, 5%. The murine mammary adenocarcinoma cell line PY8119 and PY230 were obtained from ATCC and cultured in Ham's F12 Kaighn's medium, 5% FBS, the PY230 cell line was also cultured in the presence of 0.1% MITO+ Serum Extender (Corning). The human cell line HeLa and mouse cell line LLC2 were obtained from ATCC and cultured in Eagle's Minimum Essential Medium supplemented with 10% fetal calf serum. The ID8 cell line was provided by Dr K. Roby at KUMC University of Kansas, USA and cultured in DMEM supplemented with 10% FCS.

### 1.3 In Vitro Citrullination

The citrullination of Cytokeratin 8 was performed in 0.1 M Tris-HCl PH 7.5 (Fisher), 10 mM CaCl₂) (Sigma) and 5 mM DTT (Sigma). Final concentration of solution for was 376 mM Tris-HCl PH 7.5, 3.76 mM CaCl₂), 1.88 mM DTT. Samples were incubated with PAD enzymes for 2 hrs at 37° C. before storing at −80° C. overnight or until use. PAD2 enzyme was used at a final concentration of 148 mU and PAD4 at a final concentration of 152 mU. PAD enzymes were purchased from Modiquest at 37 mU/μl hPAD2 and 38 mU/μl hPAD4.

### 1.4. Immunogens

### 1.4.1. Peptides

Peptides >90% purity were synthesized by Peptide Synthetics (Fareham, UK) and stored lyophilised in 0.2 mg aliquots at −80° C. On day of use they were reconstituted to the appropriate concentration in 10% dimethyl formamide.

### 1.5 Plasmids and Transfections

To generate the HHDII plasmid, cDNA was synthesized from total RNA isolated from EL4-HHD cells. This was used as a template to amplify HHD using the forward and reverse primers and sub cloned into pCR2.1. The HHD chain, comprising of a human HLA-A2 leader sequence, the human 32-microglobulin (B2M) molecule covalently linked via a glycine serine linker to the α 1 and 2 domains of human HLA-A*0201 MHC class I molecule and the α3, transmembrane and cytoplasmic domains of the murine H-2 Db class I molecule, was then inserted into the EcoRV/HindIII sites of the mammalian expression vector pCDNA3.1 obtained from Invitrogen.

Endotoxin free plasmid DNA was generated using the endofree Qiagen maxiprep kit (Qiagen, Crawley).

Cell lines were transfected using the Lipofectamine Transfection Reagent (Invitrogen) utilising the protocol previously described (Brentville et al. 2016). B16F1 cells were knocked out for murine MHC-I and/or MHC-II using ZFN technology (Sigma) and transfected with constitutive HLA-DP4 using the pVitro 2 chimeric plasmid. Cells were also transfected with the HHDII plasmid comprising of a human HLA-A2 leader sequence, the human B2-microglobulin (B2M) molecule covalently linked via a glycine serine linker to the α 1 and 2 domains of human HLA-0201 MHC class 1 molecule and the α3, transmembrane and cytoplasmic domains of the murine H-2 Db class 1 molecule, where relevant as previously described (Xue et al. 2016). B16F1 HHDII cells were also transfected with the pVITRO2 Human HLA-DP4 plasmid and the IFNγ inducible plasmid pDCGAS Human HLA-DP4 is described previously (Brentville et al. 2019).

### 1.6 Western Blots

Cell lysates were prepared in RIPA buffer containing protease inhibitor cocktail (Sigma) and proteins separated on a 4-12% NuPAGE Bis-Tris gel (Invitrogen) followed by transfer onto PVDF membrane. Recombinant Cytokeratin 8 protein was used as a positive control (ab73641, Abcam). The membrane was blocked for 1 hour with 3% BSA then probed with antibodies to human Cytokeratin 8 (clone TROMA-1, Millipore) used at 0.5 μg/ml and β actin (Ab8227, Abcam) 1 in 15000. Proteins were visualised using the fluorescent secondary antibody IRDye 800RD and IRDye 680RD secondary anti-mouse (for β actin). Membranes were imaged using a Licor Odyssey scanner.

### 1.7 Immunisations

### 1.7.1. Immunisation Protocol

The HHDII/HLA-DP4 transgenic strain of mouse as described in patent WO2013/017545 A1 (EMMA repository, France) and C57BL/6 mice (Charles River, UK) were used, aged between 8 and 12 weeks, and cared for by the staff at Nottingham Trent University. All work was carried out under a Home Office project licence. Peptides were dissolved in 10% dimethylformamide to 1 mg/mL and then emulsified (a series of dilutions) with the adjuvant CpG and MPLA 6 μg/mouse of each (Invivogen, UK). Peptides (25 μg/mouse) were injected subcutaneously at the base of the tail.

For tumour challenge experiments, mice were challenged with 1×10⁵ B16 HHDII/iDP4 cells subcutaneously on the right flank 3 days before primary immunisation (unless stated otherwise) and then immunised as described above. Tumour growth was monitored at 3-4 days intervals and mice humanely euthanised once tumour reached ≥10 mm in diameter.

### 1.8 Analysis of Immune Responses

### 1.8.1 Isolation and Analysis of Animal Tissue

Spleens were disaggregated and treated with red cell lysis buffer for 2 mins. Tumours were harvested and mechanically disaggregated.

### 1.8.2 Peripheral Blood Mononuclear Cell (PBMC) Isolation

Peripheral blood samples were drawn into lithium heparin tubes (Becton Dickinson) and processed immediately following venepuncture. PBMCs were isolated by density gradient centrifugation using Ficoll-Hypaque. Proliferation and cultured ELISpot assay of PBMCs were performed immediately after isolation.

### 1.8.3 Ex Vivo ELISpot Assay

ELISpot assays were performed using murine IFNγ capture and detection reagents according to the manufacturer's instructions (Mabtech, Sweden). In brief, anti-IFNγ antibody was coated onto wells of a 96-well Immobilin-P plate. Synthetic peptides (at a variety of concentrations) and 5×10⁵ per well splenocytes were added to the wells of the plate in triplicate. LPS at 5 μg/mL was used as a positive control. Peptide pulsed target cells were added where relevant at 5×10⁴ per well in triplicate and plates incubated for 40 hours at 37° C. After incubation, captured IFNγ was detected by a biotinylated anti-IFNγ antibody and developed with a streptavidin alkaline phosphatase and chromogenic substrate. Lipopolysaccharide (LPS; 5 μg/mL) was used as a positive control. For blocking studies, anti-CD4 blocking antibody (RPA-T4) and anti-CD8 blocking antibody (2.43) from Bioxcell were used at 20 μg/mL. Spots were analysed and counted using an automated plate reader (Cellular Technologies Ltd).

### 1.9 Proliferation Assay

Peripheral blood sample (approx. 50 mL) was drawn into lithium heparin tubes (Becton Dickinson). Samples were maintained at room temperature and processed immediately following venepuncture. PBMCs were isolated by density gradient centrifugation using Ficoll-Hypaque. Proliferation assay of PBMCs were performed immediately after PBMC isolation.

The median number of PBMCs routinely derived from healthy donor samples was 1.04×10⁶ PBMC/mL whole blood (range: 0.6×10⁶-1.48×10⁶/mL). The median viability as assessed by trypan blue exclusion was 93% (range 90-95%).

Freshly isolated PBMCs were loaded with carboxyfluorescein succinimidyl ester (CFSE) (ThermoFisher). Briefly, a 50 μM stock solution in warm PBS was prepared from a master solution of 5 mM in DMSO. CFSE was rapidly added to PBMCs (5×10⁶ cells/mL loading buffer (PBS with 5% v/v heat inactivated FCS)) to achieve a final concentration of 5 μM. PBMCs were incubated at room temperature in the dark for 5 mins after which non-cellular incorporated CFSE was removed by washing twice with excess (×10 v/v volumes) of loading buffer (300×g for 10 minutes). Cells were made up in complete media to 1.5×10⁶/mL and plated and stimulated with media containing vehicle (negative control), PHA (positive control, final concentration 10 μg/mL) or peptides (10 μg/mL) as described above. A donor's responses to a peptide was considered real if proliferation was double the control and greater than 1%.

On day 7-11, 500 μL of cells were removed from culture, washed in PBS and stained with 1:50 dilution of anti-CD4 (PE-Cy5, clone RPA-T4, ThermoFisher), anti-CD8 efluor 450, clone RPA-T8, ThermoFisher) and anti-CD134 (PE-Cy7, Clone REA621, Miltenyi). Cells were washed, fixed and permeabilized using intracellular fixation/permeablization buffers (ThermoFisher) according to the manufacturer's instructions. Intracellular staining for cytokines was performed using a 1:50 dilution of anti-IFNγ (clone 4S.B3, ThermoFisher) or anti-Granzyme B (PE, Clone GB11, Thermofisher). Stained samples were analysed on a MACSQuant 10 flow cytometer equipped with MACSQuant software version 2.8.168.16380 using stained vehicle stimulated controls to determine suitable gates.

### 1.10 Phenotyping T Cell Populations

Proliferation assay was performed following the method described in section 1.9 of the method section (above). On day 7-11, 500 μL of cells were removed from culture, washed in PBS and stained with 1:50 dilution of anti-CD4 (PE-Cy5, clone RPA-T4, ThermoFisher), anti-CD45RA (VioGreen, clone REA562, Miltenyi), anti CD177 (CCR7, PE-Vio770, clone REA108, Miltenyi), anti CD127 (APC-Vio770, clone REA614, Miltenyi) according to the manufacturer's instructions. Stained samples were analysed on a MACSQuant 10 flow cytometer equipped with MACSQuant software version 2.8.168. 16380 using stained vehicle stimulated controls to determine suitable gates.

### 1.11 FACS Cell Sorting

On day 10, the contents of the culture wells were mixed gently, pooled (according to peptide stimulation) and washed in PBS (300×g for 10 minutes). Pellets were gently re-suspended in 500 μL of PBS containing 10 μL of anti CD4 eFluo450 (clone RPA-T4, ThermoFisher, cat no 48-0049-42) and 10 μL of anti-CD8 APC (clone RPA-T8, ThermoFisher, cat 17-0088-41). Cells were stained at 4° C. for 30 minutes before being washed (300×g for 5 minutes) in 1 mL of PBS and resuspended in 300 μL of FACS sorting buffer (PBS supplemented with 1 mM EDTA, 25 mM HEPES and 1% v/v HI FCS). 10 μL of sample was removed from each stained sample and 90 μL of FACS sorting buffer added. 10,000 events were collected on a MACSQuant Analyser 10 flow cytometer to determine proliferation. The remaining cells were used for bulk FACS sorting.

Cells are sorted using sterile conditions in a MoFlo XDP High Speed Cell Sorter machine. All samples are sorted into 1 mL of RNA protect (5 parts Protect, Qiagen: 1-part FACS sorting buffer, Sigma) separating the CD4+ve/CFSEhigh and CD4+ve/CFSE^{low} populations. Sorted cells (bulk) are stored at −80° C.

Determination of the α and β chain pairing of TCRs recognising Cytokeratin 8 peptides containing citrulline. Sorted cells (bulk) from CD4+ve/CFSEhigh and CD4+ve/CFSElow populations in RNA protect are shipped to iRepertoire Inc (Huntsville, AL, USA) for NGS sequencing of the TCRA and TCRB chain to confirm expansion of TCR's in the CD4+ve/CFSElow cells, proliferating to the peptide in contrast to the non-proliferating CD4+ve/CFSEhigh population. In brief RNA is purified from sorted cells, RT-PCR is performed, cDNA is then subjected to Amplicon rescued multiplex PCR (ARM-PCR) using human TCR α and β 250 PER primers (iRepertoire Inc., Huntsville, AL, USA). Information about the primers can be found in the United States Patent and Trademark Office (U.S. Pat. Nos. 7,999,092 and 9,012,148B2). After assessment of PCR/DNA samples, 10 sample libraries were pooled and sequenced using the Illumina MiSeq platform (Illumina, San Diego, CA, USA). The raw data was analysed using IRweb software (iRepertoire). V, D, and J gene usage and CDR3 sequences were identified and assigned and tree maps generated using iRweb tools. Tree maps show each unique CDR3 as a coloured rectangle, the size of each rectangle corresponds to each CDR3 abundance within the repertoire and the positioning is determined by the V region usage.

To elucidate the cognate pairing and sequencing of TCRα and TCRβ chains IRepertoire use their iPair™ technology, CD4+ve/CFSElow populations of cells (bulk sorted, that were simultaneously bulk sequenced) are seeded at 1 cell/well into an iCapture 96 well plate. RT-PCR is performed and the TCRα and β chains can be amplified from the single cells using Amplicon rescued multiplex PCR (arm-PCR). Data can be analysed utilising the iPair™ Software program for frequency of specific chain pairing and the sequences ranked on comparison to bulk data.

### 1.12 Statistical Methods

Data were expressed as the number of spots per million splenocytes. Means and standard deviations (SD) were calculated from the quadruplicate readings. Means and SDs were also calculated for each group of three mice. Where appropriate Anova analysis was performed using GraphPad Prism 6 software.

### Example 1—Homology of Cytokeratin 8 Between Different Species

Two different isoforms of Cytokeratin 8 exist, isoform 1 (P05787-1) and isoform 2 (P05787-2) the peptides described are found in both isoforms (FIG. 1A). Cytokeratin 8 is highly conserved between, mouse, dog, sheep, cows, horse, pig and humans (FIG. 1B). As the vaccine induces T cell responses in humans and mice, and anti-tumour responses in mice, it can be assumed similar responses will be seen in other species.

### Example 2—T Cell Responses in HHDII/DP4 and C57BL/6 Mice to Cytokeratin 8 Epitopes

T cell responses to tumour associated epitopes are often weak or non-existent due to tolerance and T cell deletion within the thymus. The citrullinated Cytokeratin 8 peptides were screened in C57BL/6 and HHDII/DP4 transgenic mice for their ability to stimulate IFNγ responses. The selected peptides are summarised in Table 2 along with their predicted IEDB binding scores and results from the peptide screening ELISpot assays.

TABLE 2

| | | Per- | | Predic- | |
|---|---|---|---|---|---|
| | | cen- | | tion | |
| Coor- | | tile | Predicted | core | T cell |
| dinates | Sequence | score | core | rank | response |
| 8-26 | KSYKVSTSGP | 51.5 | PRAFSSRSY | 34 | Y |
| | RAFSSRSYT | | (SEQ ID | | (C57BL/ |
| | (SEQ ID | | NO: 39) | | 6) |
| | NO: 3) | | YKVSTSGPR | 69 | |
| | | | (SEQ ID | | |
| | | | NO: 17) | | |
| 101-120 | KFASFIDKVR | 15.5 | IDKVRFLEQ | 26 | Y |
| | FLEQQNKMLE | | (SEQ ID | | |
| | (SEQ ID | | NO: 18) | | |
| | NO: 1) | | | | |
| 133-151 | RSNMDNMFES | 4.25 | MFESYINNL | 58 | Y |
| | YINNLRRQL | | (SEQ ID | | |
| | (SEQ ID | | NO: 19) | | |
| | NO: 5) | | | | |
| 217-236 | LTDEINFLRQ | 13 | LRQLYEEEI | 14 | Y |
| | LYEEEIRELQ | | (SEQ ID | | |
| | (SEQ ID | | NO: 40) | | |
| | NO: 6) | | FLRQLYEEE | 12 | |
| | | | (SEQ ID | | |
| | | | NO: 20) | | |
| 371-388 | LREYQELMNV | 42 | REYQELMNV | 41 | Y |
| | KLALDIEI | | (SEQ ID | | |
| | (SEQ ID | | NO: 41) | | |
| | NO: 2) | | LMNVKLALD | 43 | |
| | | | (SEQ ID | | |
| | | | NO: 21) | | |
| 381-399 | KLALDIEIAT | 33.5 | IEIATYRKL | 42 | Y |
| | YRKLLEGEE | | (SEQ ID | | |
| | (SEQ ID | | NO: 22) | | |
| | NO: 4) | | | | |

### Screening of Cytokeratin 8 Peptide Responses

HHDII/DP4 and C57BL/6 mice were immunised with human citrullinated peptides in combination with CpG/MPLA as an adjuvant. 25 μg of peptide was administered subcutaneously as a single immunisation given once a week for three weeks Mice were culled 7 days after the third immunisation, the immune response to each peptide was assessed by ex vivo IFNγ ELISpot (FIG. 2). We have previously shown that citrullinated peptides can induce responses in the transgenic DR4 mouse strain. Given that different mouse strains have different MHC repertoires, the transgenic strain HHDII/DP4 and non-transgenic C57BL/6 mice were used for screening.

Significant IFNγ responses were detected to human Cyk8 citrullinated peptides in transgenic HHDII/DP4 mice (FIG. 2A) and C57BL/6 mice (FIG. 2B). In the HHDII/DP4 mice the Cyk8 citrullinated peptides 101-120, 133-151, 217-236, 371-388, 381-399 induced significant immune responses (p=<0.01, p=<0.001, p=<0.0001, p=<0.0001, p=<0.0001 respectively). In C57BL/6 mice only the Cyk8 8-26 citrullinated peptide induced a significant response (p=<0.001), no other significant responses were detected against any other citrullinated Cyk8 peptides in these mice. The avidity of these T cell responses in HHDII/DP4 was determined by performing a peptide titration using peptides Cyk8 133-151 cit, Cyk8 217-236 cit, Cyk8 371-388 cit (FIG. 2C). The highest avidity T cell response was seen against peptide Cyk8 371-388 cit with a log EC50 value-7.1 to-7.6 M, the avidity was slightly lower for the Cyk8 133-151 cit peptide with a log EC50 value-6.0 to-6.5 M, a slightly lower avidity was seen for the Cyk8 217-236 cit peptide with a log EC50 value-5.0 to-5.9.

To determine if the immune response to the Cyk8 peptides is specific to the citrullinated peptide and not the wild type (wt) version, HHDII/DP4 transgenic mice were immunised with Cyk8 101-120 cit, 133-151 cit, 217-236 cit, 371-388 cit, 381-399 cit or Cyk8 101 wt, 133-151 wt, 371-388 wt peptides. HHDII/DP4 mice received 25 μg peptide with CpG/MPLA subcutaneously once a week for three weeks. Mice were culled 7 days after the third immunisation, the immune response to each peptide was assessed by ex vivo ELISpot (FIGS. 3A and 3B). Moderate to high IFNγ responses were detected in mice immunised with Cyk8 101-120 cit, 133-151cit, 217-236 cit, 371-388 cit, 381-399 cit, these were significant when compared to the responses to the wt peptide (p=0.0454, p=0.0087, p=0.0003, p=0.0022, p=0.0238 respectively). Low to moderate IFNγ responses were detected in mice immunised with Cyk8 101-120 wt, Cyk8 131-15 wt, Cyk8 371-388 wt, there were no significant responses when comparing the responses to the cit and wt peptides. This confirmed that the immune response generated in HHDII/DP4 mice is in response to the citrullinated version of Cyk8 101-120, Cyk8 133-151, Cyk8 217-236, Cyk8 371-388, Cyk8 381-399 peptides and no cross-reactivity to the wt versions was observed.

The sequence of the Cyk8 101-120, 133-151, 371-388 peptides is completely homologous between humans and mice, however, the Cyk8 217-236 and Cyk8 8-26 peptides have 2 amino acid mismatches, Cyk8 381-399 and Cyk8 8-26 have 1 amino acid mismatch (FIG. 4A). To determine if the immune response to the mouse Cyk8 peptide 217-236 (mismatch at position 227 and 228) is the same as the human Cyk8 peptide HHDII/DP4 transgenic mice were immunised with mouse Cyk8 217-236cit peptide. HHDII/DP4 mice received 25 μg peptide subcutaneously in combination with CpG/MPLA as an adjuvant, once a week for three weeks. Mice were culled 7 days after the third immunisation, the immune response to the mouse and human peptide 217-236 was assessed by ex vivo ELISpot (FIG. 4B). Moderate IFNγ responses were detected in mice immunised with mouse 217-236 cit peptide, the response cross reacted to the human 217-236 cit, no significant difference was observed.

There is a high degree of sequence similarities between the different cytokeratin's. A peptide blast search was performed to determine if any of the Cytokeratin 8 peptides identified here can be found in any other cytokeratin or other antigens of interested e.g. vimentin. The results show that Cyk8 381-399 (KLALDIEIATYRKLLEGEE (SEQ ID NO: 4)) peptide was also found in Cytokeratin 4 and Glial fibrillary protein with a perfect sequence match, the Cyk8 381-399 peptide was also found in Vimentin with a 1 amino acid difference at position 2 (FIG. 5). The Cyk8 101-120 (KFASFIDKVRFLEQQNKMLE (SEQ ID NO: 1)) peptide was also found in Cytokeratin 2 and Cytokeratin 7 with a 1 amino acid difference at position 18 (FIG. 5). In addition to Cytokeratin 8 these peptides can be used to target cytokeratin 2, 4, 7, Vimentin and Glial fibrillary protein.

Previously citrullinated peptide specific responses have been shown to be CD4 mediated. To determine whether the response to Cyk8 8-26 cit, Cyk8 101-120 cit, Cyk8 133-151 cit, Cyk8 371-388 cit, Cyk8 381-399 cit is CD4 dependent HHDII/DP4 transgenic mice and C57BL/6 mice received 25 μg peptide subcutaneously in combination with CpG/MPLA as an adjuvant, once a week for three weeks (FIG. 6A HHDII/DP4, FIG. 6B C57BL/6). Mice were culled 7 days after the third immunisation, an ELISpot assay was performed in the presence of an anti CD4 or CD8 antibody. IFNγ responses were significantly reduced in the presence of the anti CD4 antibody for the Cyk8 8-26 cit (p=<0.5), Cyk8 101-120 cit (p=<0.01), Cyk8 371-388 cit (p=<0.001) and Cyk8 381-399cit (p=<0.001) peptides, the responses were not significantly reduced for the Cyk8 133-151 cit peptide. For the Cyk8 8-26 cit, Cyk8 101-120 cit, Cyk8 133-151 cit, Cyk8 371-388 cit peptides IFNγ responses were not significantly reduced in the presence of the CD8 antibody confirming that these peptides are CD4 peptides. The IFNγ response to the Cyk8 133-151 cit peptide was reduced in the presence of the anti CD4 antibody, however this did not reach significance. The IFNγ response to the Cyk8 381-399 cit peptide was significantly reduced in the presence of the anti CD8 antibody (p=<0.01) and anti CD4 antibody (p=<0.001) confirming that this peptide is CD4 (MHC class II) restricted but also likely contains a nested CD8 (MHC class I) restricted peptide.

To determine if there is a pre-existing memory population of T cells specific to the Cyk8 371-388 cit peptide HHDII/DP4 transgenic mice received 25 μg peptide subcutaneously in combination with CpG/MPLA at 14 days or 2 days prior to splenocyte harvest (FIG. 7). Moderate to high IFNγ responses were detected in mice immunised with Cyk8 371-388 cit peptide 14 days prior splenocyte harvest, these were significant when compared to the control (p=0.0163). No responses were detected in mice immunised with Cyk8 371-388 cit peptide 2 days prior splenocyte harvest. These results indicate that responses to the Cyk8 371-388 cit peptide in HHDII/DP4 transgenic mice are originating from naïve T cells and not from a pre-existing memory population which would have been boosted by an immunisation 2 days prior to harvest.

### Example 3-Cit Cytokeratin 8 Peptides Presented on Tumour Cells can be Targeted for Tumour Therapy

The inventors had already established by Western blotting that the melanoma B16F1 cell line constitutively expresses Cytokeratin 8 (FIG. 8A) and in vitro both PAD2 and PAD4 can citrullinate Cytokeratin 8 (FIG. 8B).

The effect of immunisation with Cyk8 371-388 (cit and wt) on the growth of the mouse B16 melanoma cell line transfected with IFNγ inducible human DP4 (iDP4) was assessed in HHDII/DP4 transgenic mice (FIG. 9A). The Cyk8 371-388 cit peptide was selected because it is homologous in humans and mice, it also induced the strongest immune response that is mediated by CD4 T cells. Mice were challenged with B16 HHDII/iDP4 tumour cells 3 days prior to immunisation with Cyk8 371-388 wt or Cyk8 371-388 cit. Mice immunised with Cyk8 371-388 cit peptide or Cyk8 371-388 wt showed a significant survival advantage over unimmunised control mice (p=0.0003, p=0.0018 respectively, FIG. 9A). Control mice showed 10% survival at 60 days whereas Cyk8 371-388 cit immunised mice showed 70% survival and Cyk8 371-388 wt immunised mice showed 40% survival. These results suggest that the Cyk8 371-388 wt peptide can also stimulate an anti tumour response in HHDII/DP4 transgenic mice which is not significantly different (p=0.2953) to the anti tumour response induced by the Cyk8 371-388 cit peptide but immunising with Cyk8 371-388 cit does result in a 30% improvement in survival compared to immunising with the wt peptide. There was no associated toxicity. The tumour volumes at day 29 post tumour implant was also significantly lower (p=0.0013) in the Cyk8 371-388 cit immunised mice (FIG. 9B, median 0 mm³) compared to the control group (median 509 mm³). The tumour volume was also significantly lower (p=0.0015) in the Cyk8 371-388 wt immunised mice (median 4 mm³) compared to the control group (median 509 mm³). Tumour volumes for the duration of the study show that the unimmunised mice developed larger tumours relatively quickly when compared to mice immunised with the Cyk8 371-388 cit or Cyk8 371-388 wt peptide (FIG. 9C). These results show that the Cyk8 371-388 cit peptide can induce an anti tumour response in tumour bearing HHDII/DP4 transgenic mice.

The effect of immunisation with Cyk8 101-120 cit on the growth of the mouse B16 melanoma cell line transfected with IFNγ inducible human DP4 (iDP4) was assessed in HHDII/DP4 transgenic mice (FIG. 10A). The Cyk8 101-120 cit peptide was selected because it is homologous in humans and mice, it also induced a strong immune response that is mediated by CD4 T cells. Mice were challenged with B16 HHDII/iDP4 tumour cells 3 days prior to immunisation with the Cyk8 101-120 cit peptide. Mice immunised with Cyk8 101-120 cit peptide showed a significant survival advantage over unimmunised control mice (p=0.0072, FIG. 10A). Control mice showed 20% survival at day 50 whereas Cyk8 101-120 cit immunised mice showed 70% survival on day 50. There was no associated toxicity. The tumour volumes at day 24 post tumour implant was also significantly lower (p=0.0221) in the Cyk8 101-120 cit immunised mice (FIG. 10B, median 2 mm³) compared to the control group (median 89 mm³). The tumour volumes for the duration of the study show that the unimmunised mice had a larger tumour volume (FIG. 10C, highest median 509 mm³ on day 29) when compared with the immunised group (FIG. 10C, highest median 9 mm³ on days 35 and 38). These results show that the Cyk8 101-120 cit peptide can induce an anti tumour response in tumour bearing HHDII/DP4 transgenic mice.

The effect of immunisation with Cyk8 8-26 cit on the growth of the mouse B16 melanoma cell line was assessed in C57BL/6. Mice were challenged with B16 tumour cells 3 days prior to immunisation with Cyk8 8-26 cit. Mice immunised with Cyk8 8-26 cit peptide showed a significant survival advantage over unimmunised control mice (p=0.0002, FIG. 11A). Control mice showed 0% survival at 20 days whereas Cyk8 8-26 cit immunised mice showed 70% survival on day 20, this further reduced to 40% survival by day 29. There was no associated toxicity. The tumour volumes at day 13 post tumour implant was also significantly lower (p=0.0054) in the Cyk8 8-26 cit immunised mice (FIG. 11B, median 19 mm³) compared to the control group (median 586 mm³). The overall tumour volumes for the duration of the study show that the unimmunised mice had larger tumour volumes (FIG. 11C, highest median 1150 mm³ on day 17) when compared with the immunised group (FIG. 11C, highest median 523 mm³ on day 23). These results show that the Cyk8 8-26 cit peptide can induce an anti tumour response in tumour bearing C57BL/6 mice.

### Example 4-Responses to Cytokeratin 8 in Healthy Human Donors and Cancer Patients

In HHDII/DP4 mice, the response to Cyk8 371-388 cit peptide could not be detected 2 days post immunisation but could be detected 14 days after immunisation. This suggests that these are naive responses and no pre-existing immunity exists in these mice. This raised the question of whether humans have or can generate immune responses to citrullinated Cyk8 peptides. For this study the response to Cyk8 101-120 cit peptide was determined in healthy donors and cancer patients, the clinical details of the ovarian and lung cancer patients are listed in tables 3 and 4 respectively. To investigate this, PBMC's were isolated from eighteen healthy donors and cultured in the presence of Cyk8 101-120 cit peptide. Thirteen donors were HLA-DP4 positive, two donors were HLA-DP4 negative, for three donors the HLA type could not be determined.

TABLE 3

| Donor code | Disease | Treatment |
|---|---|---|
| OV8 | Stage 4 papillary serous | Finished 6 cycles of |
| | ovarian carcinoma. | carboplatin + paclitaxel |
| | | (paclitaxel omitted for the |
| | | last 2 cycles) |
| OV9 | Stage 4 high grade serous | Finished 6 cycles of |
| | ovarian carcinoma. | carboplatin + paclitaxel + |
| | | bevacizumab but continues |
| | | on maintenance bevacizumab. |
| OV10 | Stage 4 high grade serous | On tamoxifen. Not had prior |
| | ovarian carcinoma. | chemotherapy. |
| OV12 | Stage 3C high grade | Finished 4 cycles of |
| | serous ovarian carcinoma. | carboplatin + paclitaxel + |
| | | bevacizumab 3 weeks prior to |
| | | blood sample. Surgery |
| | | abandoned due to extensive |
| | | disease intra-operatively. |
| OV13 | Stage 4 high grade papillary | Treatment naïve. |
| | serous ovarian carcinoma. | |
| OV14 | Stage 4 high grade serous | Finished 6 cycles of |
| | ovarian carcinoma. | carboplatin + paclitaxel |
| | | (first 2 cycles only single |
| | | agent carboplatin) but |
| | | continued on maintenance |
| | | bevacizumab |
| OV15 | Stage 3C high grade | Finished 6 cycles of |
| | papillary serous ovarian | rechallenge carboplatin + |
| | carcinoma. | paclitaxel. Remained on |
| | | Niraparib. |
| OV17 | Stage 3C high grade | Treatment naïve. |
| | serous ovarian carcinoma. | |
| OV18 | Stage 3C ovarian | Finished 4 cycles of |
| | endometrioid | carboplatin + gemcitabine |
| | adenocarcinoma. | |
| OV19 | Stage 3C low grade serous | Blood sample taken 2 months |
| | ovarian adenocarcinoma. | following ultra-radical surgery |
| | | and prior to starting adjuvant |
| | | chemotherapy. |
| OV21 | Stage 3C high grade | finished 6 cycles of |
| | papillary serous ovarian | carboplatin and caelyx, had |
| | carcinoma, BRCA-ve | partial response to treatment |
| | | on CT scan. |
| OV22 | Patient with stage 4 | Treatment naïve |
| | primary peritoneal | |
| | adenocarcinoma | |

TABLE 4

| Donor code | Disease | Treatment |
|---|---|---|
| LG1 | Metastatic adenocarcinoma of | Carboplatin and |
| | lung) | pemetrexed |
| LG2 | Metastatic small cell lung | Finished |
| | cancer | chemoradiotherapy. |
| LG10 | T4N2M1a adenocarcinoma of | Pembrolizumab |
| | lung and previously resected | |
| | renal cell carcinoma | |
| LG9 | Metastatic adenocarcinoma of | Carboplatin and |
| | lung | pemetrexed |
| LG13 | T4N3MO poorly differentiated | Pembrolizumab |
| | non-small cell lung cancer | |
| | (NSCLC) | |

PBMCs from eighteen healthy donors were labelled with Carboxyfluorescein succinimidyl ester (CFSE) prior to in vitro culture in the presence of Cyk8 101-120 cit peptide. On day 7 and 10 cells were stained with anti-CD4 and anti-CD8 fluorochrome conjugated antibodies, proliferation was then assessed by flow cytometry (FIG. 12A). On day 7, a CD4 Cyk8 101-120 cit specific proliferating (CFSE^{low}) population could be detected in four out of eighteen donors, this increased at day 10 with six out of eighteen donors showing a specific response (FIG. 12B). On day 10, functional analysis was performed on five out of the six donors that showed a good CD4 Cyk8 101-120 cit specific proliferative response. The expression of IFNγ, Granzyme B and CD134 was determined on specific proliferating T cells from donors where proliferative responses were observed (FIG. 12C, D and E). The proliferating CD4 Cyk8 101-120 cit specific T cells from all five donors expressed CD134 (above media control background), in addition; four out of five donors also expressed IFNγ and two out of five expressed Granzyme B. This data shows that in healthy donors T cells responses can be detected in response to the Cyk8 101-120 cit peptides, these T cells proliferate, and express markers associated with functionality.

PBMCs from five lung cancer patients and twelve ovarian cancer patients were labelled with Carboxyfluorescein succinimidyl ester (CFSE) prior to in vitro culture in the presence of Cyk8 101-120 cit peptide. On day 7 and 10, cells were stained with anti-CD4 and anti-CD8 fluorochome conjugated antibodies, proliferation was then assessed by flow cytometry (FIG. 13). On day 7, a CD4 Cyk8 101-120 cit specific proliferating (CFSE^{low}) population could be detected in one out of five lung cancer patients. This frequency decreased at day 10 with none of the lung cancer patient T cells showing a specific response (FIG. 13A). On day 7, Cyk8 101-120 cit peptide specific proliferating (CFSE^{low}) population could be detected in three out of twelve ovarian cancer patients. This increased on day 10 with four out of twelve ovarian cancer patients T cells showing a specific response to Cyk8 101-120 cit peptide (FIG. 13B). Functional analysis was performed on day 7 and 10 for one lung cancer patient and four ovarian cancer patients (FIGS. 13C and 12D) that showed a good CD4 Cyk8 101-120 cit specific proliferative response. The proliferating CD4 Cyk8 101-120 cit specific T cells from the lung cancer patient did not show any expression of CD134 (above media control background), IFNγ or CD134 (FIG. 13C), only one out of four ovarian cancer patients showed specific expression of CD134 (above media control background) and Granzyme B but not IFNγ (FIG. 13D). This data shows that in cancer patients, T cells responses can be detected in response to the Cyk8 101-120 cit peptides in a few patients, but these are low in frequency and magnitude.

These results suggest that T cells from healthy donors are able to generate a CD4 proliferative response to the Cyk8 101-120 cit peptide which is also associated with the upregulation of functional markers associated with cytotoxic activity. PBMCs from a small number of cancer patients are also able to generate a CD4 response to Cyk8 101-120 cit peptide, although the frequency was low. The proliferative magnitude of the Cyk8 101-120 cit peptide specific T cell response was lower in the lung cancer and ovarian cancer patients compared to the healthy donors. This lower magnitude of T cell responses in cancer patients could be due to medication they are on or some degree of tumour mediated immune suppression in these patients. These results show that a lower number of cancer patients respond to the Cyk8 101-120 cit peptide when compared to healthy donors, in the majority of cancer patients that showed a Cyk8 101-120 cit specific proliferative response there was no co-expression of functional markers.

### Example 5-In Healthy Human Donors Naive T Cell Populations Respond to Cyk8 101-120 Cit Peptide

PBMCs from one healthy donor were labelled with Carboxyfluorescein succinimidyl ester (CFSE) prior to in vitro culture in the presence of Cyk8 101-120 cit peptide. On day 10, cells were stained with anti-CD4, anti CD45RA, anti CD177 (CCR7) and anti CD127 proliferation and the phenotype of the responding T cells was then assessed by flow cytometry (FIG. 14). On day 10, a CD4 Cyk8 101-120 cit specific proliferating population could be detect, the majority of these T cells were memory cells (5.96% TEMRA, 52.91% effector memory, 32.75% central memory) and only 8.38% were naive T cells. These results show that in healthy donors a Cyk8 371-388 cit specific T cell response can be generated in healthy donors following peptide stimulation, the majority of the responding T cells are memory T cells.

### REFERENCES

Altschul, S. F., W. Gish, W. Miller, E. W. Myers, and D. J. Lipman. 1990. ‘Basic local alignment search tool’, J Mol Biol, 215:403-10.
Altschul, S. F., T. L. Madden, A. A. Schaffer, J. Zhang, Z. Zhang, W. Miller, and D. J. Lipman. 1997. ‘Gapped BLAST and PSI-BLAST: a new generation of protein database search programs’, Nucleic Acids Res, 25:3389-402.
Andreatta, M., V. I. Jurtz, T. Kaever, A. Sette, B. Peters, and M. Nielsen. 2017. ‘Machine learning reveals a non-canonical mode of peptide binding to MHC class II molecules’, Immunology, 152:255-64.
Arnold, P. Y., N. L. La Gruta, T. Miller, K. M. Vignali, P. S. Adams, D. L. Woodland, and D. A. Vignali. 2002. ‘The majority of immunogenic epitopes generate CD4+ T cells that are dependent on MHC class II-bound peptide-flanking residues’, J Immunol, 169:739-49.
Arstila, T. P., A. Casrouge, V. Baron, J. Even, J. Kanellopoulos, and P. Kourilsky. 1999. ‘A direct estimate of the human alphabeta T cell receptor diversity’, Science, 286:958-61. Ausubel, J M. 1992. Short protocols in molecular biology (John Wiley & Sons).
Bird, R. E., K. D. Hardman, J. W. Jacobson, S. Johnson, B. M. Kaufman, S. M. Lee, T. Lee, S. H. Pope, G. S. Riordan, and M. Whitlow. 1988. ‘Single-chain antigen-binding proteins’, Science, 242:423-6.
Blobel, Gerd A., Roland Moll, Werner W. Franke, and Ingolf Vogt-Moykopf. 1984. ‘Cytokeratins in normal lung and lung carcinomas’, Virchows Archiv B, 45:407-29.
Bluemke, K., U. Bilkenroth, A. Meye, S. Fuessel, C. Lautenschlaeger, S. Goebel, A. Melchior, H. Heynemann, P. Fornara, and H. Taubert. 2009. ‘Detection of circulating tumor cells in peripheral blood of patients with renal cell carcinoma correlates with prognosis’, Cancer Epidemiol Biomarkers Prev, 18:2190-4.
Bodanzsky, Bodanzsky &. 1984. The practice of peptide synthesis (Springer Verlag: New York). Boulter, J. M., M. Glick, P. T. Todorov, E. Baston, M. Sami, P. Rizkallah, and B. K. Jakobsen. 2003. ‘Stable, soluble T-cell receptor molecules for crystallization and therapeutics’, Protein Eng, 16:707-11.
Brentville, V. A., R. L. Metheringham, B. Gunn, P. Symonds, I. Daniels, M. Gijon, K. Cook, W. Xue, and L. G. Durrant. 2016. ‘Citrullinated Vimentin Presented on MHC-II in Tumor Cells Is a Target for CD4+ T-Cell-Mediated Antitumor Immunity’, Cancer Res, 76:548-60.
Brentville, V. A., P. Symonds, K. W. Cook, I. Daniels, T. Pitt, M. Gijon, P. Vaghela, W. Xue, S. Shah, R. L. Metheringham, and L. G. Durrant. 2019. ‘T cell repertoire to citrullinated self-peptides in healthy humans is not confined to the HLA-DR SE alleles; Targeting of citrullinated self-peptides presented by HLA-DP4 for tumour therapy’, Oncoimmunology, 8: e1576490. Brown, J. H., T. S. Jardetzky, J. C. Gorga, L. J. Stern, R. G. Urban, J. L. Strominger, and D. C. Wiley. 1993. ‘Three-dimensional structure of the human class II histocompatibility antigen HLA-DR1’, Nature, 364:33-9.
Carson, R. T., K. M. Vignali, D. L. Woodland, and D. A. Vignali. 1997. ‘T cell receptor recognition of MHC class II-bound peptide flanking residues enhances immunogenicity and results in altered TCR V region usage’, Immunity, 7:387-99.
Chang, X., Y. Zhao, Y. Wang, Y. Chen, and X. Yan. 2013. ‘Screening citrullinated proteins in synovial tissues of rheumatoid arthritis using 2-dimensional western blotting’, J Rheumatol, 40:219-27.
Chen, N., J. Gong, X. Chen, M. Xu, Y. Huang, L. Wang, N. Geng, and Q. Zhou. 2009. ‘Cytokeratin expression in malignant melanoma: potential application of in-situ hybridization analysis of mRNA’, Melanoma Res, 19:87-93.
Chou, Y. H., O. Skalli, and R. D. Goldman. 1997. ‘Intermediate filaments and cytoplasmic networking: new connections and more functions’, Curr Opin Cell Biol, 9:49-53.
Choy, E. 2012. ‘Understanding the dynamics: pathways involved in the pathogenesis of rheumatoid arthritis’, Rheumatology (Oxford), 51 Suppl 5: v3-11.
Chung, B. M., J. D. Rotty, and P. A. Coulombe. 2013. ‘Networking galore: intermediate filaments and cell migration’, Curr Opin Cell Biol, 25:600-12.
Coimbra, S., A. Figueiredo, E. Castro, P. Rocha-Pereira, and A. Santos-Silva. 2012. ‘The roles of cells and cytokines in the pathogenesis of psoriasis’, Int J Dermatol, 51:389-95; quiz 95-8. Consogno, G., S. Manici, V. Facchinetti, A. Bachi, J. Hammer, B. M. Conti-Fine, C. Rugarli, C. Traversari, and M. P. Protti. 2003. ‘Identification of immunodominant regions among promiscuous HLA-DR-restricted CD4+ T-cell epitopes on the tumor antigen MAGE-3’, Blood, 101:1038-44.
Corper, A. L., T. Stratmann, V. Apostolopoulos, C. A. Scott, K. C. Garcia, A. S. Kang, I. A. Wilson, and L. Teyton. 2000. ‘A structural framework for deciphering the link between I-Ag7 and autoimmune diabetes’, Science, 288:505-11.
Coulombe, P. A., and M. B. Omary. 2002. “Hard’ and ‘soft’ principles defining the structure, function and regulation of keratin intermediate filaments', Curr Opin Cell Biol, 14:110-22.
Davis, M. M., J. J. Boniface, Z. Reich, D. Lyons, J. Hampl, B. Arden, and Y. Chien. 1998. ‘Ligand recognition by alpha beta T cell receptors’, Annu Rev Immunol, 16:523-44.
Dessen, A., C. M. Lawrence, S. Cupo, D. M. Zaller, and D. C. Wiley. 1997. ‘X-ray crystal structure of HLA-DR4 (DRA*0101, DRB1*0401) complexed with a peptide from human collagen II’, Immunity, 7:473-81.
Douat-Casassus, C., N. Marchand-Geneste, E. Diez, N. Gervois, F. Jotereau, and S. Quideau. 2007. ‘Synthetic anticancer vaccine candidates: rational design of antigenic peptide mimetics that activate tumor-specific T-cells’, J Med Chem, 50:1598-609.
Durrant, L. G., R. L. Metheringham, and V. A. Brentville. 2016. ‘Autophagy, citrullination and cancer’, Autophagy, 12:1055-6.
Folch, G., D. Scaviner, V. Contet, and M. P. Lefranc. 2000. ‘Protein displays of the human T cell receptor alpha, beta, gamma and delta variable and joining regions’, Exp Clin Immunogenet, 17:205-15.
Franke, W. W., D. L. Schiller, R. Moll, S. Winter, E. Schmid, I. Engelbrecht, H. Denk, R. Krepler, and B. Platzer. 1981. ‘Diversity of cytokeratins. Differentiation specific expression of cytokeratin polypeptides in epithelial cells and tissues’, J Mol Biol, 153:933-59.
Fremont, D. H., W. A. Hendrickson, P. Marrack, and J. Kappler. 1996. ‘Structures of an MHC class II molecule with covalently bound single peptides’, Science, 272:1001-4.
Fremont, D. H., D. Monnaie, C. A. Nelson, W. A. Hendrickson, and E. R. Unanue. 1998. ‘Crystal structure of I-Ak in complex with a dominant epitope of lysozyme’, Immunity, 8:305-17.
Fuchs, E., and D. W. Cleveland. 1998. ‘A structural scaffolding of intermediate filaments in health and disease’, Science, 279:514-9.
Fukunaga, Y., S. Bandoh, J. Fujita, Y. Yang, Y. Ueda, S. Hojo, K. Dohmoto, Y. Tojo, J. Takahara, and T. Ishida. 2002. ‘Expression of cytokeratin 8 in lung cancer cell lines and measurement of serum cytokeratin 8 in lung cancer patients’, Lung Cancer, 38:31-8.
Garboczi, D. N., D. T. Hung, and D. C. Wiley. 1992. ‘HLA-A2-peptide complexes: refolding and crystallization of molecules expressed in Escherichia coli and complexed with single antigenic peptides’, Proc Natl Acad Sci USA, 89:3429-33.
Gebauer, M., and A. Skerra. 2009. ‘Engineered protein scaffolds as next-generation antibody therapeutics’, Curr Opin Chem Biol, 13:245-55.
Ghosh, P., M. Amaya, E. Mellins, and D. C. Wiley. 1995. ‘The structure of an intermediate in class II MHC maturation: CLIP bound to HLA-DR3’, Nature, 378:457-62.
Godkin, A. J., K. J. Smith, A. Willis, M. V. Tejada-Simon, J. Zhang, T. Elliott, and A. V. Hill. 2001. ‘Naturally processed HLA class II peptides reveal highly conserved immunogenic flanking region sequence preferences that reflect antigen processing rather than peptide-MHC interactions’, J Immunol, 166:6720-7.
Gonzalez-Galarza, F. F., L. Y. Takeshita, E. J. Santos, F. Kempson, M. H. Maia, A. L. da Silva, A. L. Teles e Silva, G. S. Ghattaoraya, A. Alfirevic, A. R. Jones, and D. Middleton. 2015. ‘Allele frequency net 2015 update: new features for HLA epitopes, KIR and disease and HLA adverse drug reaction associations’, Nucleic Acids Res, 43: D784-8.
Green, D. R., and B. Levine. 2014. ‘To be or not to be? How selective autophagy and cell death govern cell fate’, Cell, 157:65-75.
Greten, T. F., and J. P. Schneck. 2002. ‘Development and use of multimeric major histocompatibility complex molecules’, Clin Diagn Lab Immunol, 9:216-20.
Grunewald, J., and A. Eklund. 2007. ‘Role of CD4+ T cells in sarcoidosis’, Proc Am Thorac Soc, 4:461-4.
Gu, L. H., and P. A. Coulombe. 2007. ‘Keratin function in skin epithelia: a broadening palette with surprising shades’, Curr Opin Cell Biol, 19:13-23.
Holliger, P., and G. Winter. 1993. ‘Engineering bispecific antibodies’, Curr Opin Biotechnol, 4:446-9. Holmdahl, R., L. Klareskog, K. Rubin, E. Larsson, and H. Wigzell. 1985. ‘T lymphocytes in collagen II-induced arthritis in mice. Characterization of arthritogenic collagen II-specific T-cell lines and clones’, Scand J Immunol, 22:295-306.
Hoppes, R., R. Oostvogels, J. J. Luimstra, K. Wals, M. Toebes, L. Bies, R. Ekkebus, P. Rijal, P. H. Celie, J. H. Huang, M. E. Emmelot, R. M. Spaapen, H. Lokhorst, T. N. Schumacher, T. Mutis, B. Rodenko, and H. Ovaa. 2014. ‘Altered peptide ligands revisited: vaccine design through chemically modified HLA-A2-restricted T cell epitopes’, J Immunol, 193:4803-13.
Hosken, N. A., and M. J. Bevan. 1990. ‘Defective presentation of endogenous antigen by a cell line expressing class I molecules’, Science, 248:367-70.
Huston, J. S., D. Levinson, M. Mudgett-Hunter, M. S. Tai, J. Novotny, M. N. Margolies, R. J. Ridge, R. E. Bruccoleri, E. Haber, R. Crea, and et al. 1988. ‘Protein engineering of antibody binding sites: recovery of specific activity in an anti-digoxin single-chain Fv analogue produced in Escherichia coli’, Proc Natl Acad Sci USA, 85:5879-83.
Ireland, J. M., and E. R. Unanue. 2011. ‘Autophagy in antigen-presenting cells results in presentation of citrullinated peptides to CD4 T cells’, J Exp Med, 208:2625-32. June, C. H., M. V. Maus, G. Plesa, L. A. Johnson, Y. Zhao, B. L. Levine, S. A. Grupp, and D. L. Porter. 2014. ‘Engineered T cells for cancer therapy’, Cancer Immunol Immunother, 63:969-75. Jurtz, V., S. Paul, M. Andreatta, P. Marcatili, B. Peters, and M. Nielsen. 2017. ‘NetMHCpan-4.0: Improved Peptide-MHC Class I Interaction Predictions Integrating Eluted Ligand and Peptide Binding Affinity Data’, J Immunol, 199:3360-68.
Karantza, V. 2011. ‘Keratins in health and cancer: more than mere epithelial cell markers’, Oncogene, 30:127-38.
Karlin, S., and S. F. Altschul. 1993. ‘Applications and statistics for multiple high-scoring segments in molecular sequences’, Proc Natl Acad Sci USA, 90:5873-7.
Katsuragi, K., M. Yashiro, T. Sawada, H. Osaka, M. Ohira, and K. Hirakawa. 2007. ‘Prognostic impact of PCR-based identification of isolated tumour cells in the peritoneal lavage fluid of gastric cancer patients who underwent a curative RO resection’, Br J Cancer, 97:550-6.
Kim, A., I. Z. Hartman, B. Poore, T. Boronina, R. N. Cole, N. Song, M. T. Ciudad, R. R. Caspi, D. Jaraquemada, and S. Sadegh-Nasseri. 2014. ‘Divergent paths for the selection of immunodominant epitopes from distinct antigenic sources’, Nat Commun, 5:5369.
Knosel, T., V. Emde, K. Schluns, P. M. Schlag, M. Dietel, and I. Petersen. 2006. ‘Cytokeratin profiles identify diagnostic signatures in colorectal cancer using multiplex analysis of tissue microarrays’, Cell Oncol, 28:167-75.
Latek, R. R., A. Suri, S. J. Petzold, C. A. Nelson, O. Kanagawa, E. R. Unanue, and D. H. Fremont. 2000. ‘Structural basis of peptide binding and presentation by the type I diabetes-associated MHC class II molecule of NOD mice’, Immunity, 12:699-710.
Lee, K. H., K. W. Wucherpfennig, and D. C. Wiley. 2001. ‘Structure of a human insulin peptide-HLA-DQ8 complex and susceptibility to type 1 diabetes’, Nat Immunol, 2:501-7.
Lefranc, M. P. 2001. ‘Nomenclature of the human T cell receptor genes’, Curr Protoc Immunol, Appendix 1: Appendix 1O. 2003. ‘IMGT databases, web resources and tools for immunoglobulin and T cell receptor sequence analysis, http://imgt.cines.fr’, Leukemia, 17:260-6.
Li, Y., H. Li, R. Martin, and R. A. Mariuzza. 2000. ‘Structural basis for the binding of an immunodominant peptide from myelin basic protein in different registers by two HLA-DR2 proteins’, J Mol Biol, 304:177-88.
Li, Y., R. Moysey, P. E. Molloy, A. L. Vuidepot, T. Mahon, E. Baston, S. Dunn, N. Liddy, J. Jacob, B. K. Jakobsen, and J. M. Boulter. 2005. ‘Directed evolution of human T-cell receptors with picomolar affinities by phage display’, Nat Biotechnol, 23:349-54.
Liao, J., N. O. Ku, and M. B. Omary. 1997. ‘Stress, apoptosis, and mitosis induce phosphorylation of human keratin 8 at Ser-73 in tissues and cultured cells’, J Biol Chem, 272:17565-73.
Liddy, N., G. Bossi, K. J. Adams, A. Lissina, T. M. Mahon, N. J. Hassan, J. Gavarret, F. C. Bianchi, N. J. Pumphrey, K. Ladell, E. Gostick, A. K. Sewell, N. M. Lissin, N. E. Harwood, P. E. Molloy, Y. Li, B. J. Cameron, M. Sami, E. E. Baston, P. T. Todorov, S. J. Paston, R. E. Dennis, J. V. Harper, S. M. Dunn, R. Ashfield, A. Johnson, Y. McGrath, G. Plesa, C. H. June, M. Kalos, D. A. Price, A. Vuidepot, D. D. Williams, D. H. Sutton, and B. K. Jakobsen. 2012. ‘Monoclonal TCR-redirected tumor cell killing’, Nat Med, 18:980-7.
Matros, E., G. Bailey, T. Clancy, M. Zinner, S. Ashley, E. Whang, and M. Redston. 2006. ‘Cytokeratin 20 expression identifies a subtype of pancreatic adenocarcinoma with decreased overall survival’, Cancer, 106:693-702.
McCormack, E., K. J. Adams, N. J. Hassan, A. Kotian, N. M. Lissin, M. Sami, M. Mujic, T. Osdal, B. T. Gjertsen, D. Baker, A. S. Powlesland, M. Aleksic, A. Vuidepot, O. Morteau, D. H. Sutton, C. H. June, M. Kalos, R. Ashfield, and B. K. Jakobsen. 2013. ‘Bi-specific TCR-anti CD3 redirected T-cell targeting of NY-ESO-1- and LAGE-1-positive tumors’, Cancer Immunol Immunother, 62:773-85.
Mertz, K. D., F. Demichelis, A. Sboner, M. S. Hirsch, P. Dal Cin, K. Struckmann, M. Storz, S. Scherrer, D. M. Schmid, R. T. Strebel, N. M. Probst-Hensch, M. Gerstein, H. Moch, and M. A. Rubin. 2008. ‘Association of cytokeratin 7 and 19 expression with genomic stability and favorable prognosis in clear cell renal cell cancer’, Int J Cancer, 123:569-76.
Moll, R., M. Divo, and L. Langbein. 2008. ‘The human keratins: biology and pathology’, Histochem Cell Biol, 129:705-33.
Moll, R., W. W. Franke, D. L. Schiller, B. Geiger, and R. Krepler. 1982. ‘The catalog of human cytokeratins: patterns of expression in normal epithelia, tumors and cultured cells’, Cell, 31:11-24.
Munz, C. 2012. ‘Antigen Processing for MHC Class II Presentation via Autophagy’, Front Immunol, 3:9.
Myers, E. W., and W. Miller. 1989. ‘Approximate matching of regular expressions’, Bull Math Biol, 51:5-37.
Nygren, P. A. 2008. ‘Alternative binding proteins: affibody binding proteins developed from a small three-helix bundle scaffold’, FEBS J, 275:2668-76.
Oshima, R. G. 2002. ‘Apoptosis and keratin intermediate filaments’, Cell Death Differ, 9:486-92. 2007. ‘Intermediate filaments: a historical perspective’, Exp Cell Res, 313:1981-94.
Oshima, R. G., H. Baribault, and C. Caulin. 1996. ‘Oncogenic regulation and function of keratins 8 and 18’, Cancer Metastasis Rev, 15:445-71.
Owens, D. W., and E. B. Lane. 2003. ‘The quest for the function of simple epithelial keratins’, Bioessays, 25:748-58.
Paramio, J. M., and J. L. Jorcano. 2002. ‘Beyond structure: do intermediate filaments modulate cell signalling?’, Bioessays, 24:836-44.
Pearson, W. R., and D. J. Lipman. 1988. ‘Improved tools for biological sequence comparison’, Proc Natl Acad Sci USA, 85:2444-8.
Pluckthun, A. 1991. ‘Antibody engineering: advances from the use of Escherichia coli expression systems’, Biotechnology (N Y), 9:545-51.
Reff, M. E. 1993. ‘High-level production of recombinant immunoglobulins in mammalian cells’, Curr Opin Biotechnol, 4:573-6.
Remington, R P. 1980. Remington's pharmaceutical sciences (Mack Pub. Co).
Robins, H. S., P. V. Campregher, S. K. Srivastava, A. Wacher, C. J. Turtle, O. Kahsai, S. R. Riddell, E. H. Warren, and C. S. Carlson. 2009. ‘Comprehensive assessment of T-cell receptor beta-chain diversity in alphabeta T cells’, Blood, 114:4099-107.
Sambrook, J, Fritsch and Maniatis. 1989. Molecular cloning: A laboratory manual (Cold Spring Harbor Laboratory Press).
Schmid, D., M. Pypaert, and C. Munz. 2007. ‘Antigen-loading compartments for major histocompatibility complex class II molecules continuously receive input from autophagosomes’, Immunity, 26:79-92.
Schmitz-Winnenthal, F. H., C. Volk, B. Helmke, S. Berger, U. Hinz, M. Koch, J. Weitz, J. Kleeff, H. Friess, M. Zoller, M. W. Buchler, and K. Z′Graggen. 2006. ‘Expression of cytokeratin-20 in pancreatic cancer: an indicator of poor outcome after RO resection’, Surgery, 139:104-8.
Scott, C. A., P. A. Peterson, L. Teyton, and I. A. Wilson. 1998. ‘Crystal structures of two I-Ad-peptide complexes reveal that high affinity can be achieved without large anchor residues’, Immunity, 8:319-29.
Sette, A., L. Adorini, S. M. Colon, S. Buus, and H. M. Grey. 1989. ‘Capacity of intact proteins to bind to MHC class II molecules’, J Immunol, 143:1265-7.
Skerra, A. 2008. ‘Alternative binding proteins: anticalins-harnessing the structural plasticity of the lipocalin ligand pocket to engineer novel binding activities’, FEBS J, 275:2677-83.
Smith, K. J., J. Pyrdol, L. Gauthier, D. C. Wiley, and K. W. Wucherpfennig. 1998. ‘Crystal structure of HLA-DR2 (DRA*0101, DRB1*1501) complexed with a peptide from human myelin basic protein’, J Exp Med, 188:1511-20.
Soeth, E., U. Grigoleit, B. Moellmann, C. Roder, B. Schniewind, B. Kremer, H. Kalthoff, and I. Vogel. 2005. ‘Detection of tumor cell dissemination in pancreatic ductal carcinoma patients by CK 20 RT-PCR indicates poor survival’, J Cancer Res Clin Oncol, 131:669-76.
Stefansson, I. M., H. B. Salvesen, and L. A. Akslen. 2006. ‘Loss of p63 and cytokeratin 5/6 expression is associated with more aggressive tumors in endometrial carcinoma patients’, Int J Cancer, 118:1227-33.
Stern, L. J., J. H. Brown, T. S. Jardetzky, J. C. Gorga, R. G. Urban, J. L. Strominger, and D. C. Wiley. 1994. ‘Crystal structure of the human class II MHC protein HLA-DR1 complexed with an influenza virus peptide’, Nature, 368:215-21.
Stewart. 1984. Solid phase peptide synthesis (Illinois Pierce Chemical Company: Rockford).
Szeverenyi, I., A. J. Cassidy, C. W. Chung, B. T. Lee, J. E. Common, S. C. Ogg, H. Chen, S. Y. Sim, W. L. Goh, K. W. Ng, J. A. Simpson, L. L. Chee, G. H. Eng, B. Li, D. P. Lunny, D. Chuon, A. Venkatesh, K. H. Khoo, W. H. McLean, Y. P. Lim, and E. B. Lane. 2008. ‘The Human Intermediate Filament Database: comprehensive information on a gene family involved in many human diseases’, Hum Mutat, 29:351-60.
Thomas, P. A., D. A. Kirschmann, J. R. Cerhan, R. Folberg, E. A. Seftor, T. A. Sellers, and M. J. Hendrix. 1999. ‘Association between keratin and vimentin expression, malignant phenotype, and survival in postmenopausal breast cancer patients’, Clin Cancer Res, 5:2698-703.
Thomsen, M. C., and M. Nielsen. 2012. ‘Seq2Logo: a method for construction and visualization of amino acid binding motifs and sequence profiles including sequence weighting, pseudo counts and two-sided representation of amino acid enrichment and depletion’, Nucleic Acids Res, 40: W281-7.
Torelli, A., and C. A. Robotti. 1994. ‘ADVANCE and ADAM: two algorithms for the analysis of global similarity between homologous informational sequences’, Comput Appl Biosci, 10:3-5. Traunecker, A., A. Lanzavecchia, and K. Karjalainen. 1991. ‘Bispecific single chain molecules (Janusins) target cytotoxic lymphocytes on HIV infected cells’, EMBO J, 10:3655-9.
Trill, J. J., A. R. Shatzman, and S. Ganguly. 1995. ‘Production of monoclonal antibodies in COS and CHO cells’, Curr Opin Biotechnol, 6:553-60.
Vaidya, M. M., A. M. Borges, S. A. Pradhan, R. M. Rajpal, and A. N. Bhisey. 1989. ‘Altered keratin expression in buccal mucosal squamous cell carcinoma’, J Oral Pathol Med, 18:282-6.
Wang, X., P. Chen, J. Cui, C. Yang, and H. Du. 2015. ‘Keratin 8 is a novel autoantigen of rheumatoid arthritis’, Biochem Biophys Res Commun, 465:665-9.
Ward, E. S., D. Gussow, A. D. Griffiths, P. T. Jones, and G. Winter. 1989. ‘Binding activities of a repertoire of single immunoglobulin variable domains secreted from Escherichia coli’, Nature, 341:544-6.
Watt, P. M. 2006. ‘Screening for peptide drugs from the natural repertoire of biodiverse protein folds’, Nat Biotechnol, 24:177-83.
Xue, W., R. L. Metheringham, V. A. Brentville, B. Gunn, P. Symonds, H. Yagita, J. M. Ramage, and L. G. Durrant. 2016. ‘SCIB2, an antibody DNA vaccine encoding NY-ESO-1 epitopes, induces potent antitumor immunity which is further enhanced by checkpoint blockade’, Oncoimmunology, 5: e1169353.
Yang, X. R., Y. Xu, G. M. Shi, J. Fan, J. Zhou, Y. Ji, H. C. Sun, S. J. Qiu, B. Yu, Q. Gao, Y. Z. He, W. Z. Qin, R. X. Chen, G. H. Yang, B. Wu, Q. Lu, Z. Q. Wu, and Z. Y. Tang. 2008. ‘Cytokeratin 10 and cytokeratin 19: predictive markers for poor prognosis in hepatocellular carcinoma patients after curative resection’, Clin Cancer Res, 14:3850-9.
Zhu, X., H. J. Belmont, S. Price-Schiavi, B. Liu, H. I. Lee, M. Fernandez, R. L. Wong, J. Builes, P. R. Rhode, and H. C. Wong. 2006. ‘Visualization of p53 (264-272)/HLA-A*0201 complexes naturally presented on tumor cell surface by a multimeric soluble single-chain T cell receptor’, J Immunol, 176:3223-32.

1. A citrullinated T cell antigen which comprises, consists essentially of, or consists of
i) one or more of the amino acid sequences:
KFASFIDKVRFLEQQNKMLE
LREYQELMNVKLALDIEI
KSYKMSTSGPRAFSSRSFT
KLALDIEIATYRKLLEGEE
RSNMDNMFESYINNLRRQL
and
LTDEINFLRQLYEEEIRELQ,
wherein at least one arginine (R) residue in the sequence is replaced with citrulline, and/or
ii) one or more amino acid sequences of i), with the exception of 1, 2 or 3 amino acid substitutions, and/or 1, 2 or 3 amino acid insertions, and/or 1, 2 or 3 amino acid deletions in a non-arginine position.

2. The antigen of claim 1, which comprises, consists essentially of, or consists of
i) one or more of the following amino acid sequences:
KFASFIDKV-cit-FLEQQNKMLE
L-cit-EYQELMNVKLALDIEI
KSYKMSTSGP-cit-AFSSRSFT
KLALDIEIATY-cit-KLLEGEE
cit-SNMDNMFESYINNL-cit--cit-QL
LTDEINFL-cit-QLYEEEI-cit-ELQ
wherein “cit” represents citrulline, and/or
ii) one or more of the amino acid sequences of i), with the exception of 1, 2 or 3 amino acid substitutions, and/or 1, 2 or 3 amino acid insertions, and/or 1, 2 or 3 amino acid deletions in a non-citrulline position

3. A complex of the antigen of claim 1 and an MHC molecule, optionally wherein the MHC molecule is MHC class II, optionally DP4.

4. A binding moiety that binds the antigen of claim 1.

5. The binding moiety of claim 4, which binds the antigen when it is in complex with MHC.

6. The binding moiety of claim 4, wherein the binding moiety is a T cell receptor (TCR) or an antibody.

7. The binding moiety of claim 7, wherein the TCR is on the surface of a cell.

8. An antigen as defined in claim 1 for use in medicine.

9. The antigen, complex, and/or binding moiety for use as defined in claim 8 for use in treating or preventing cancer.

10. The antigen, complex, and/or binding moiety for use as defined in claim 9, wherein the cancer is AML, lung, colorectal, renal, breast, ovary and liver tumours.

11. A pharmaceutical composition comprising an antigen as defined in claim 1, together with a pharmaceutically acceptable carrier.

12. A method of identifying a binding moiety that binds a complex as claimed in claim 3, the method comprising contacting a candidate binding moiety with the complex and determining whether the candidate binding moiety binds the complex.

13. An in vitro method of screening to identify a citrullinated T cell epitope of cytokeratin that stimulates anti-tumour immunity, comprising:
screening cytokeratin for induction of a T cell response specific to a citrullinated epitope;
and
screening T cells specific for the citrullinated epitope for tumour recognition.

14. The method of claim 13, wherein screening for induction of T cell response to a citrullinated epitope comprises sorting CD4 and CD8 T cells to identify whether the citrullinated epitope is a CD4 or CD8 epitope.

15. A complex of the antigen of claim 2, and an MHC molecule, optionally wherein the MHC molecule is MHC class II, optionally DP4.

16. The binding moiety of claim 5, wherein the binding moiety is a T cell receptor (TCR) or an antibody.

17. A pharmaceutical composition comprising an antigen of claim 2.

18. A pharmaceutical composition comprising a binding moiety of claim 4.
